(19) United States
(12) Patent Application Publication
(10) Pub. No.: US 2022/0364081 A1
(43) Pub. Date: Nov. 17, 2022

(54) MOLECULAR LIBRARY ENCODING SYSTEM AND METHODS

(71) Applicant: Pavel Chubukov, San Diego, CA (US)

(72) Inventor: Pavel Chubukov, San Diego, CA (US)

(21) Appl. No.: 17/638,161

(22) PCT Filed: Aug. 30, 2020
(86) PCT No.: PCT/US2020/048666

§ 371 Date: Feb. 24, 2022

Related U.S. Application Data
(60) Provisional application No. 62/894,683, filed on Aug. 30, 2019.

(51) Int. Cl. C12N 15/10; G01N 21/64

(52) U.S. Cl. CPC C12N 15/1065; G01N 21/6428; G01N 21/6456; B82Y 20/00

(57) ABSTRACT

The present invention provides methods and systems for encoding and decoding of synthesis steps and conditions of combinatorial synthesis of molecular library on carrier-beads. The encoding is performed at each step of synthesis by attachment of smaller fluorescently labelled beads (label-beads) to the surface of a carrier-bead (carrier-bead). The number of label-beads should be such that each is spatially resolvable on a surface of the carrier-bead. Alternatively label-beads are detachable, or the carrier-bead are dissolvable, so the label-beads could be dispersed over large enough distance to be resolved spatially. The fluorescent spectrum of each of the label-beads carries information of the synthesis step and synthesis, i.e., a spectral barcode or binary encoding system. During decoding of the spectrally identified label-beads, a fluorescent spectrum of each spatially resolvable label-bead is determined.

### BACKGROUND.

Drug discovery is a costly and specialized research area typically carried out at large research facilities. Costs in analysis and manpower are reduced with the use of high-throughput screening (HTS) of molecular libraries. A molecular library can range thousand to several million different species. The technological constraints in generation, identification and management of a molecular library is a barrier to high throughput screening of both small molecules and macromolecules. The use of bead bound library where each bead contains one kind of compound, is useful in the screening of the library for biological activity. However, the size and types of libraries that can be created and identified in a HTS with a bead based system is limited by existing labelling techniques and methods and the types of compatible reactions that can be done on the beads in conjunction with the labelling system. For example, DNA/polynucleotide encoded combinatorial libraries have proved to be useful in the drug discovery field. For example, oligonucleotide encoded libraries are disclosed in US 2019/0210018. WO2018089641 describes polynucleotide encoded chemical libraries on a bead based system where an oligonucleotide encodes a compound library member. However, the compatibility of the DNA/polynucleotide restricts the chemical space and also limits the types of reactions that can be done on the bead due to chemical sensitivity of the DNA/polynucleotide to the reaction conditions. This leads to a decrease in the quality of the compounds (incomplete reactions) and to limitations on the type of compounds that can be synthesized on the bead. Other fluorescence encoded systems for the labeling of beads are also known for encoding molecular libraries. Whereas these system libraries have nearly unlimited chemical space, their encoding capacity is limited.

Therefore, there is a need for an encoding system and for systems and methods for creating and screening molecular libraries which is cost-effective and efficient and has a high accuracy. There is also a need for an encoding system and methods that can be used with a wide variety of chemical reaction conditions to produce large molecular libraries, where the compounds in the molecular library are readily identifiable.

### SUMMARY

According to the invention, a set of carrier beads having a set of labelling beads is provided. The labelling beads have a plurality of labelling bead types, each labelling bead type being adapted to attach to the surface of a carrier bead or be captured in the bulk of a carrier bead. Each labelling bead type has a unique fluorescent label which is optically resolvable from the unique fluorescent label of every other labelling bead type when attached to the surface of a carrier bead or captured in the bulk of a carrier bead.

The unique fluorescent label on each labelling bead type has optically resolvable fluorescent bands. Preferably, the fluorescent bands comprise one, two or more fluorescence emission bands and one, two or more fluorescence excitation bands. Each fluorescence emission band is distinguishable by one, two or more of: (i) the representative wavelength of emission, (ii) the intensity of emission, (iii) the characteristic wavelength of the excitation band exciting the emission, and (iv) the efficiency of excitation in this excitation band exciting the emission. The unique fluorescent label on each labelling bead type is spectrally distinct from the unique fluorescent labels on the other labelling bead types in the set of labelling beads, such that the plurality of labelling bead types, in combination, are each uniquely labelled.

According to another embodiment of the invention, a binary encoding system for the identification of carrier beads is provided. According to the system, a number N of different labelling bead types, are provided. The N different labelling bead types are used to prepare an exponentially large number, 2^{N}, of carrier bead types, each labelling bead type being adapted to attach to the surface of a carrier bead or be captured in the bulk of a carrier bead, each labelling bead type having unique fluorescent label which is optically resolvable from the unique fluorescent label of other labelling bead type when attached to the surface of a carrier bead or captured in the bulk of a carrier bead. The unique fluorescent label on each labelling bead type is optically resolvable and spectrally distinct from the unique fluorescent label on the other labelling bead types in the set of labelling beads, as described herein.

The unique fluorescent label on at least one labelling bead type is identifiable with an optical interrogation technique, such as confocal fluorescence imaging with spectral resolution or confocal fluorescence spectroscopy. The carrier beads may be optically interrogated with three-dimensional (3D) resolution and the diameter of the smallest optically resolved 3D region is smaller than the diameter of the carrier beads. The presence or the absence of a labelling bead type on the surface or in the bulk of the carrier bead provides 1 bit of encoding for the carrier bead type. All possible combinations of beads of N different types being present or absent on the surface or in the bulk of the carrier bead provide N bits of encoding and 2^{N} unique fluorescent encodings. The availability of 10, 20, and 30 different types of labelling beads makes it possible to prepare carrier beads with, respectively, 2¹⁰≈10³, 2²⁰≈10⁶, and 2³⁰≈10⁹ unique fluorescent encodings.

According to another embodiment, a system for preparing a combinatorial library with fluorescent encoding in a combinatorial synthesis is provided. The system comprises providing a set of labelling beads comprised of a plurality of labelling bead types according to the invention described herein. A plurality of carrier beads is also provided. A set of molecular building blocks for a combinatorial synthesis is also provided. The combinatorial synthesis has a plurality of steps, and each step of the combinatorial synthesis has a molecular building block, each building block being the same or different. A sequence of steps of the combinatorial synthesis are performed on a group of carrier beads, the sequence of steps comprising adding a new molecular building block to a group of carrier beads at each step of the combinatorial synthesis to form the combinatorial library, each group of carrier beads in the library having a unique synthetic compound. Each molecular building block bound to the group carrier beads in a given sequence step of the combinatorial synthesis is matched with a labelling bead type, thereby forming a population of different carrier bead groups, each carrier bead group having a unique synthetic molecule and corresponding unique fluorescent label encoded on the carrier bead, the unique fluorescent label being produced by the plurality of different labelling bead types, each with unique fluorescent labels, attached to the surface of the carrier beads in consecutive steps of the combinatorial synthesis. The labelling bead types attached to each carrier bead are identified by an optical interrogation technique.

According to another embodiment of the invention, a combinatorial library is established on a population of fluorescently encoded carrier beads, as described herein. The fluorescently encoded carrier beads comprise a plurality of groups of carrier beads, each group of carrier beads has a synthetic molecule, the synthetic molecule having a plurality of molecular building blocks added at N consecutive steps in the combinatorial synthesis. A plurality of different labelling bead types, as described herein, are attached to the surface of the carrier beads. The labelling bead type is attached to the carrier bead at a given step of the combinatorial synthesis and uniquely matches the type of the molecular building block added to the carrier bead at the same step of the combinatorial synthesis, such that the set of labelling bead types attached to a given carrier bead uniquely fluorescently encodes the set of the molecular building blocks in the synthetic molecules on the carrier bead. All carrier beads belonging to a given group in the population of the carrier beads carry the same synthetic molecules comprised of the same sequence of molecular building blocks and have the same fluorescent encoding, whereas beads belonging to different groups carry different synthetic molecules and have different fluorescent encodings that are optically resolvable on the carrier beads with the optical interrogation techniques described herein.

According to another embodiment, a method of labelling carrier beads in a solid phase combinatorial synthesis is provided. The method comprises first providing a carrier bead having a primary compound attached thereto. Next, a set of molecular building blocks for each step of the synthesis is provided. The carrier bead having the primary compound is combined with a molecular building block to form synthetic molecule M1 that is comprised of the primary compound and the first added building block. The carrier bead with synthetic molecule M1 is attached to a plurality of first labelling bead types, the first labelling bead type being correlated to the first molecular building block to form a carrier bead having molecule M1 and label L1. Molecular building blocks are M2-M4 to the synthetic molecule M1 on the surface of the carrier bead and attaching a second labelling bead type, L2-L4 to the surface of the carrier bead to form carrier beads having synthetic molecules M1,M2,L1,L2; M1,M3,L1,L3; and M1,M4,L1,L4. Subsequent building blocks Mx and correlating labelling bead types Lx are added to the prior created synthetic molecules to form carrier beads with synthetic molecules M1,M2,Mx,L1,L2; M1,M3,Mx,L1,L3Lx; and M1,M4,Mx,L1,L4,Lx, where Mx represents the set of building blocks, and Lx represents the corresponding labelling bead type. As an end product, carrier beads compatible with the optical interrogation technique described herein are obtained, making it possible to detect the types of labelling beads attached to the surface of each carrier bead and identify the corresponding synthetic compound.

According to another embodiment of the invention, a method of decoding the composition of the synthetic molecules contained on the carrier beads described herein is provided. The method comprises providing a population of carrier beads comprised of a plurality of groups, each group of the carrier beads in the population having a unique synthetic compound on the surface and a unique fluorescent encoding provided by a unique set of types of labelling beads attached to the surface of the carrier beads. Each carrier bead of interest is optically interrogated with an optical interrogation technique. Using the results of the optical interrogation, all individual types of the labelling beads on the surface of each carrier bead of interest are identified. The newly identified types of the labelling beads are matched with the types of molecular building blocks to identify all individual molecular building blocks in the synthetic molecules on the carrier bead and the order in which these building blocks were added. Using the information on the type and order of the newly identified building blocks. The structure of the synthetic molecules on the carrier bead may be obtained.

According to another embodiment, a system for preparing a combinatorial library with binary fluorescent encoding in a combinatorial synthesis is provided. According to the method, sets of molecular building blocks used at each of the steps in a combinatorial synthesis are selected the numbers of different building blocks in the sets used at steps 1, 2, 3, n, as B1, B2, B3, Bn, respectively, are calculated. A set of labelling beads as described herein are provided and split in cohorts 1, 2, 3, n, with the number of cohorts equal to or greater than the number of steps of the combinatorial synthesis, and with the number of types of the labelling beads in cohorts 1, 2, 3, n . . . , N1, N2, N3, Nn, respectively, satisfying the equations N1≥log₂(B1), N2≥log₂(B2), N3≥log₂(B3) . . . , such that the molecular building blocks added at each step of the combinatorial synthesis can be binary encoded by the labelling beads from the corresponding cohort of types. Carrier beads as described herein are provided. A combinatorial synthesis, as described herein, is performed on each of the carrier beads to form the combinatorial with a plurality of groups of carrier beads, each group of carrier beads in the library having a unique synthetic compound. The new molecular building blocks are matched with unique labelling bead types, as described herein. The labelling bead types attached to each carrier bead and using the information on the bead types to identify the type and order of the molecular building blocks on the carrier bead to obtain the structure of the synthetic molecules on the carrier bead.

According to another embodiment, a combinatorial library established on a population of fluorescently encoded carrier beads, the fluorescently encoded carrier beads, as described herein, is provided. The combinatorial library comprises a plurality of groups of carrier beads, each group of carrier beads comprising a synthetic molecule, the synthetic molecule having a plurality of molecular building blocks added at n consecutive steps in a combinatorial synthesis, wherein the numbers of different molecular building blocks added at steps 1, 2, 3, . . . n, are B1, B2, B3 . . . Bn, respectively; and a plurality of different labelling bead types attached to the surface of the carrier beads. The numbers of labelling beads in cohorts used for steps 1, 2, 3, . . . n; are N1, N2, N3, . . . Nn, respectively, satisfying the equations N1≥log₂(B1), N2≥log₂(B2), N3≥log₂(B3) . . . , such that the molecular building blocks added at each step of the combinatorial synthesis can be binary encoded by the labelling beads from the corresponding cohort of labelling bead types, as described herein. The labelling bead types attached to each carrier bead and using the information on the bead types to identify the type and order of the molecular building blocks on the carrier bead to obtain the structure of the synthetic molecules on the carrier bead.

A method of labelling carrier beads as described herein, in a solid phase synthesis is also provided, as well as a method of screening a chemical library for molecules and macromolecules having certain desired properties is also provided. The method comprises providing a population of carrier beads with a fluorescently encoded library of molecules or macromolecules as described herein. The populations of carrier beads are assay tested for one or more testing outcomes. The population of carrier beads are optically interrogated for which the desired testing outcomes were observed. Using the results of the optical interrogation to identify the molecular structures of the synthetic molecules on the carrier beads for which the desired testing outcome of the assay was observed.

### BRIEF DESCRIPTION OF THE DRAWINGS

These and other features, aspects and advantages of the present invention will become better understood from the following description, appended claims, and accompanying figures where:

FIG. 1 illustrative spectra of exemplary label-bead systems based on quantum dots (QD) as sources of fluorescence;

FIG. 2 illustrates quantitative spectral separation of an exemplary labelling bead system based on quantum dots as sources of fluorescence, according to the present invention;

FIG. 3 illustrates a fluorescent spectral label for an exemplary labelling bead according to the present invention, showing multiple fluorescence bands at different wavelengths, and also having varying intensity of the fluorescence emission.

FIG. 4 illustrates a fluorescent spectral label for an exemplary labelling bead system for quantum dot encoding according to the present invention, where the labelling bead system has synthesis stage encoding bands and building block encoding bands;

FIG. 5A illustrates fluorescent spectra for a plurality of exemplary unique fluorescent labels for the different labelling bead types illustrated in FIG. 5B, according to the invention;

FIG. 5B is an illustration of a carrier bead having a plurality of groups of labelling bead types, each labelling bead type having a unique fluorescent label, according to the invention.

FIG. 6 is an expanded view of the exemplary fluorescence spectra shown in FIG. 5A.

FIG. 7 illustrates fluorescent spectra for a plurality of exemplary unique fluorescent labels, where the encoding is used in a binary fashion, according to another embodiment of the invention;

FIG. 8 is a graphical illustration the binary encoding system, according to the invention;

FIG. 9 illustrates combinatorial synthesis using binary spatial-spectral encoding, according to the embodiments of the invention shown in FIGS. 7 and 8.

FIG. 10 illustrates a synthesis on carrier beads using a plurality of labelling bead types (i.e., encoding QD beads), where a labelling bead type is attached to the carrier bead at each step (stage) in the synthesis, according to another embodiment of the invention;

FIG. 11 illustrates a synthesis of a small molecule on a carrier bead using a plurality of labelling bead types, where a labelling bead type is attached to the carrier bead at each step (stage) in the synthesis, as shown in FIG. 10, according to another embodiment of the invention;

FIG. 12 illustrates a synthesis of a macromolecule on a carrier bead using a plurality of labelling bead types, where a labelling bead type is attached to the carrier bead at each step (stage) in the synthesis, as shown in FIG. 10, according to the invention;

FIG. 13 illustrates the information coding capacity for an exemplary synthesis.

FIG. 14 illustrates a combinatorial synthesis using the carrier beads of the invention, where a starting molecule is attached to the carrier bead, followed by attaching a second molecule and corresponding labelling beads to the carrier bead, according to the invention;

FIG. 15 illustrates stage-n iterations of a combinatorial synthesis using the carrier beads where a starting molecule is attached to carrier beads, the group of carrier beads is split, followed by attaching second different molecules and corresponding different labelling bead types to the carrier beads corresponding to each molecule in each step of the syntheses, according to another embodiment of the invention;

FIG. 16 illustrates Stage A of a multi stage a combinatorial synthesis using the carrier beads of the invention, where a starting molecule is attached to the carrier bead, followed by attaching a second molecule and corresponding labelling beads to the carrier bead to form beads a1, according to the invention.

FIG. 17 illustrates the a combinatorial synthesis using the carrier beads where a starting molecule is attached to carrier beads, the group of carrier beads is split, followed by attaching second different molecules and corresponding different labelling bead types to the carrier beads corresponding to each molecule in each step of the syntheses, in reactions a1, a2, and a3, according to the invention;

FIG. 18 illustrates stage B of a combinatorial synthesis using the carrier beads a1, where a starting molecule is attached to carrier beads, the group of carrier beads is split, followed by attaching second and third different molecules, and corresponding different labelling bead types to the carrier beads, the different labelling bead types corresponding to each molecule in each step of the syntheses, in reactions a1, and b1, according to the invention.

FIG. 19 further illustrates stage B of a combinatorial synthesis using the carrier beads a1, where the a1 beads are split, followed by attaching third different molecules, and corresponding different labelling bead types to the carrier beads, the different labelling bead types corresponding to each different molecule in each step of the syntheses, in reactions of a1, and b1, b2, and b3, according to the invention;

FIG. 20 illustrates some of the carrier beads and molecules in stage B of an illustrative combinatorial synthesis, each carrier bead having a unique fluorescent label, according to the invention;

FIG. 21 illustrates stage C of a combinatorial synthesis where carrier beads from the reactions a1 and b1 are modified with molecules on each bead with corresponding blocks and correspondingly labeled with a differently labelling bead type, according to the invention;

FIG. 22 further illustrates stage C of a combinatorial synthesis using the carrier beads a1,b1 in a reaction with c1, c2, and c3, where the a1,b1 beads are split, followed by attaching fourth different molecules, and corresponding different labelling bead types to the carrier beads, the different labelling bead types corresponding to each different molecule in each step of the syntheses, in reactions of a1,b1 and c1, c2, and c3, according to the invention;

FIG. 23 illustrates some of the carrier beads and molecules in stage C of an illustrative combinatorial synthesis, each carrier bead having a unique fluorescent label, according to the invention; and

FIG. 24 illustrates special-spectral information decoding for the fluorescent labels according to the invention.

### DETAILED DESCRIPTION

In the present invention, dedicated label-beads, also referred to herein as labelling beads, or labelling bead types, are used to fluorescently label and encode carrier-beads for identifying molecular libraries. The invention also describes systems and methods for detection of the label-beads on the surface and in the bulk of carrier-beads. Systems for labelling a combinatorial library in a combinatorial synthesis are also provided, as well as molecular libraries based on the label-beads and carrier-beads and methods of the generation and decoding of fluorescent labels.

According to the present invention, a set of carrier-beads and a set of label-beads, for labelling the carrier-beads are provided. The label-beads and labelled carrier-beads described herein have more encoding capacity via spatially resolved units of information on the label-beads. The invention described herein allows an expanded fluorescent encoding capacity of more than 10¹² members. In the systems described herein for labelling a combinatorial library in a combinatorial synthesis, each compound in the library is made of one or more monomers and a series of chemical (molecular) building blocks that make up the library. The unique combination of label-beads on each carrier-bead is uniquely identifiable and matches with the particular compound on the carrier-bead. As all carrier-beads carrying a given molecular compound in the molecular library have the same unique fluorescent encoding (label), making the compound on each carrier-bead identifiable, the compounds can be readily assayed and screened for biological activity. The label-beads, labelled carrier-beads, systems and methods disclosed herein can be applied to high throughput screening, target discovery, or diagnostics, and other similar assays.

According to the present invention, a set of carrier-beads and a set of label-beads for labelling the set of carrier-beads is provided. Each label-bead can attach to the surface of a carrier-bead or can be captured in the bulk of a carrier-bead. The label-beads come in a plurality of types. All label-beads of a given type have a unique fluorescent label which is optically resolvable from the unique fluorescent labels on all other types of label-beads when attached to the surface of the carrier-bead or captured in the bulk of the carrier-bead. The unique fluorescent label on each type of label-bead comprises one, two or more fluorescence excitation and emission bands distinguishable by the wavelengths of fluorescence excitation and/or the wavelength and intensity of the fluorescence emission. The unique fluorescent label on each type of label-bead is selected from a larger set of fluorescence excitation and emission bands, each fluorescent excitation and emission band in the larger set being spectrally distinct from the other fluorescent excitation and emission bands in the larger set to form the plurality of types of label-beads with distinct spectra and/or intensities of fluorescence.

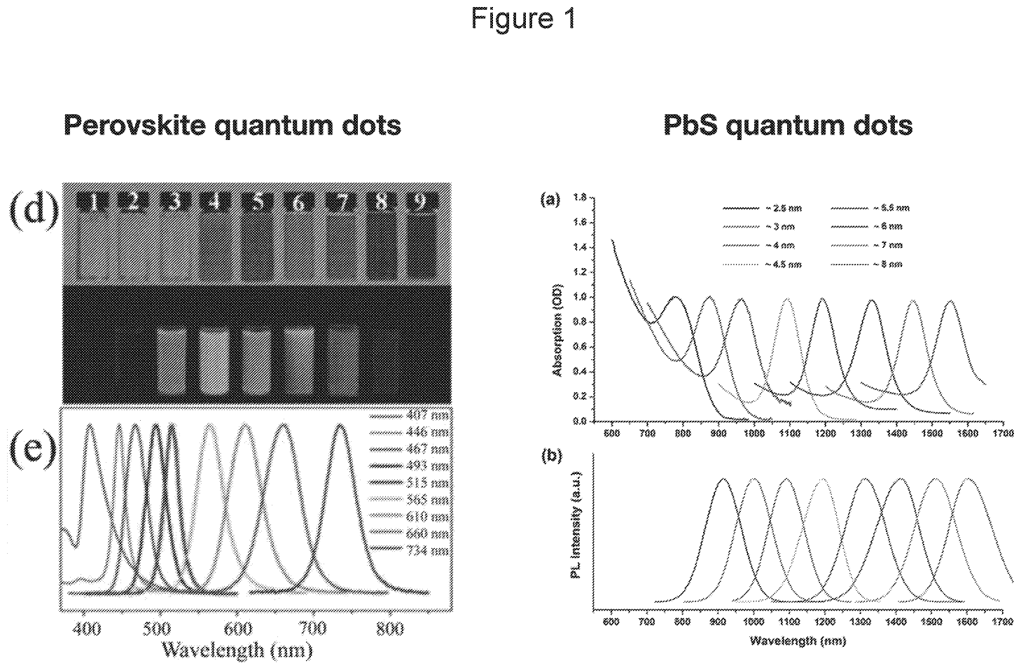

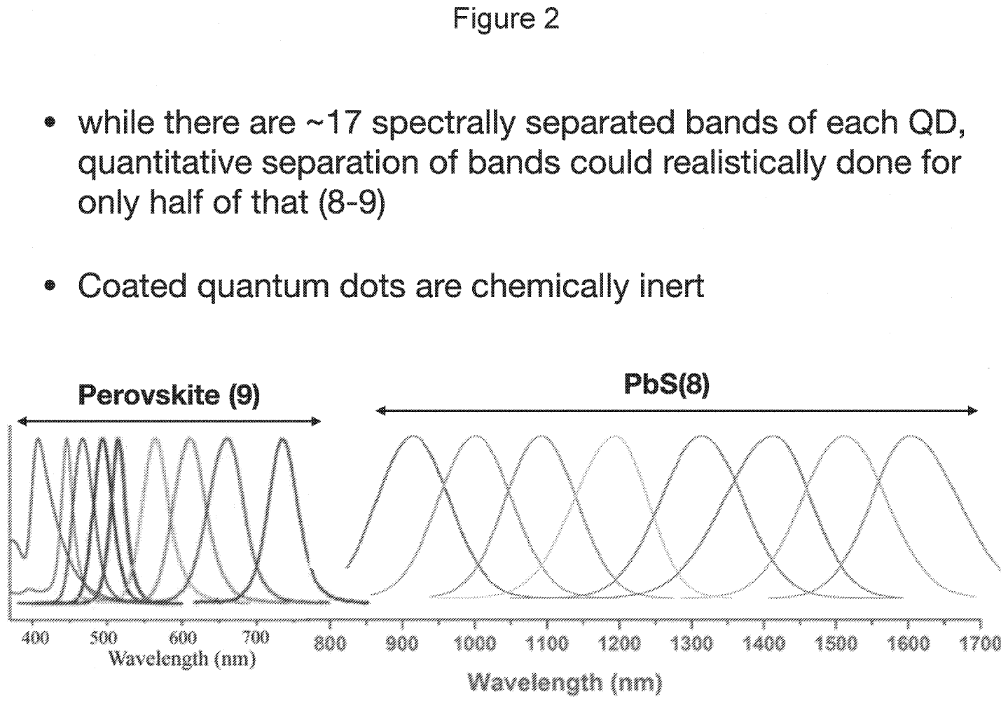

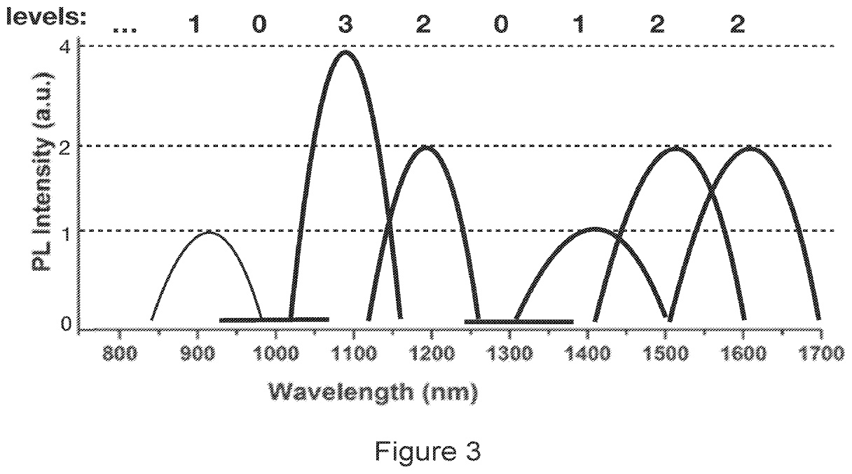

FIGS. 1-3 show an example of a label-bead system based on quantum dots (QD) as sources of fluorescence, with the emission spectra of 9 QDs in the visible or near-infrared (IR) and emission spectra of 9 QDs in 8 spectra in IR (8-9). The quantum dots are preferably encapsulated in a chemically inert material and the surfaces of label-beads and carrier-beads are functionalized to enable the attachment of label-beads to carrier-beads. A quantum dot label-bead can be created to have a unique fluorescent label which is optically resolvable from the unique fluorescent labels on all other types of label-beads when attached to the surface of the carrier-bead or captured in the bulk of the carrier-bead.

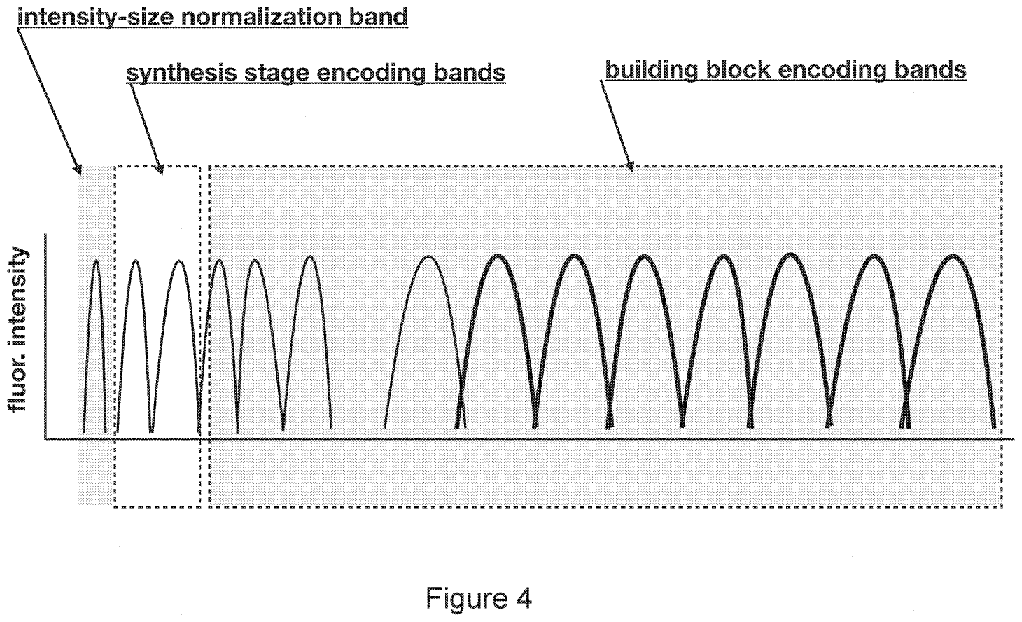

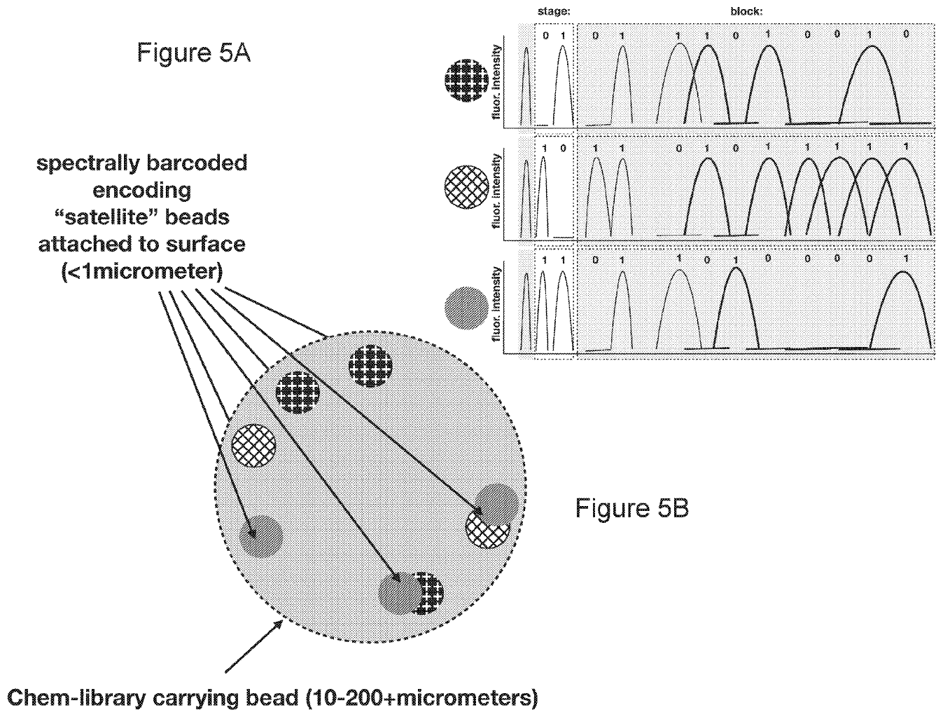

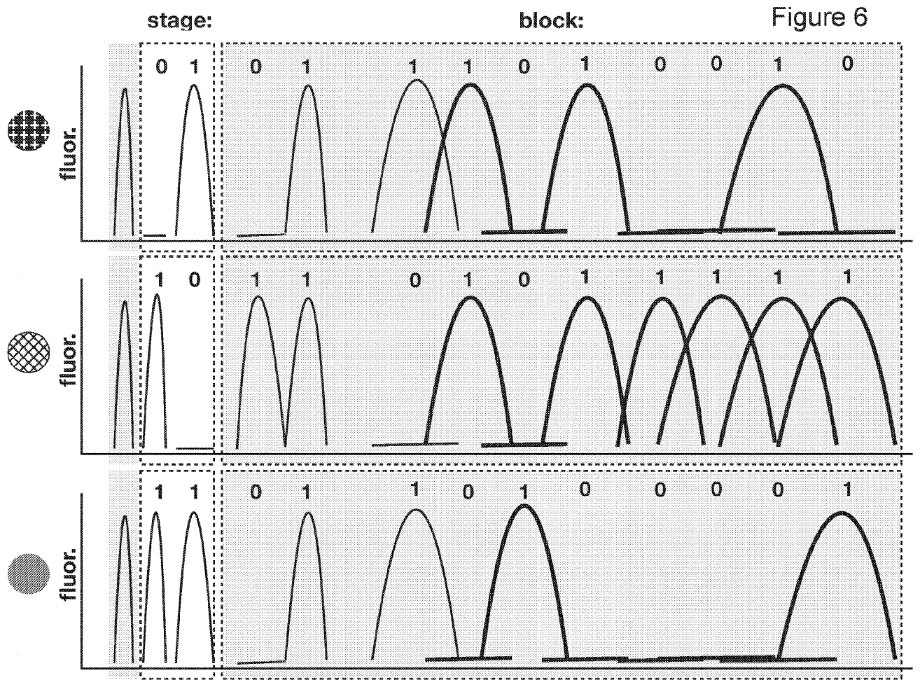

When each label-bead has a single type of QDs inside, the uses of 9 or 17 different types of QDs inside allows, respectively, for 2⁹≈500 or 2¹⁷≈130,000 unique binary encoding combinations. Critical for this encoding capability is that label-beads are individually optically resolvable when attached to a single carrier bead, making it possible to use optical interrogation to find all types of label-beads attached to the carrier bead (FIGS. 4-6). This optical interrogation technique is based on imaging with three-dimensional (3D) resolution, where the diameter of the 3D resolved region is substantially smaller than the diameter of carrier beads. FIG. 4 illustrates an exemplary fluorescence spectra for quantum dot encoding according to the present invention. FIG. 5A illustrates fluorescent spectra for a plurality of exemplary unique fluorescent labels for different labelling bead types, as illustrated in FIG. 5B. FIG. 5B is an illustration of a carrier bead having a plurality of groups of labelling bead types, each labelling bead type having a unique fluorescent label, according to the present invention. FIG. 6 is an expanded view of the exemplary fluorescence spectra shown in FIG. 5A.

According to one embodiment, each of the label-beads is encoded with two or more distinguishable fluorescence emission bands, each individual band being selected from a larger set of four or more fluorescence emission bands. Preferably, the fluorescence is derived from fluorescent quantum dots.

In some embodiments, the diameter of the carrier-beads is three or more times greater than the diameter of the label-beads.

In some embodiments, the diameter of the smallest optically resolved 3D region is 10 or more times smaller than the diameter of the carrier-beads. The optical interrogation technique allows for distinguishing between the intensities and spectra of fluorescence of individual label-beads belonging to different types when the label-beads are attached to the surface or captured in the bulk of the carrier-beads.

According to the optical interrogation techniques described herein, the optical interrogation technique can be confocal fluorescence imaging with spectral resolution or confocal fluorescence spectroscopy.

As described herein, when the optical interrogation techniques described above are used, the spectral and intensity sensitivities and the 3D spatial resolution are sufficiently high to reliably detect the type of an absolute majority of the individual label-beads attached to the surface or captured in the bulk of the carrier-bead, as to ensure that the proportion of label-beads whose type is not identified or misidentified is practically negligible. In on embodiment the diameter of the smallest resolved 3D region is smaller than the diameter of the label-beads; in another embodiment the diameter of the smallest resolved 3D region is smaller than average distance between the label-beads.

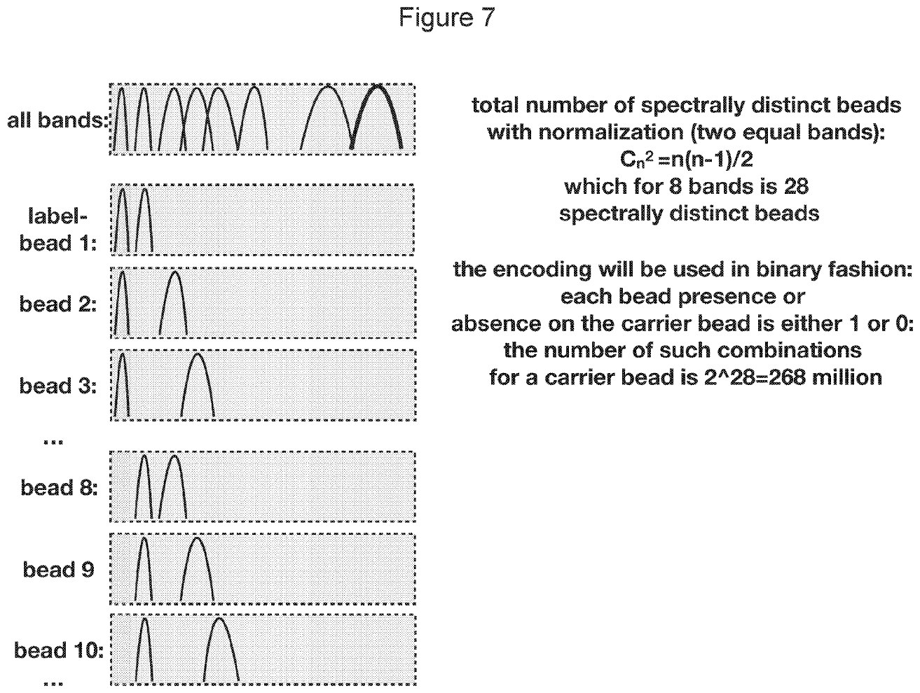

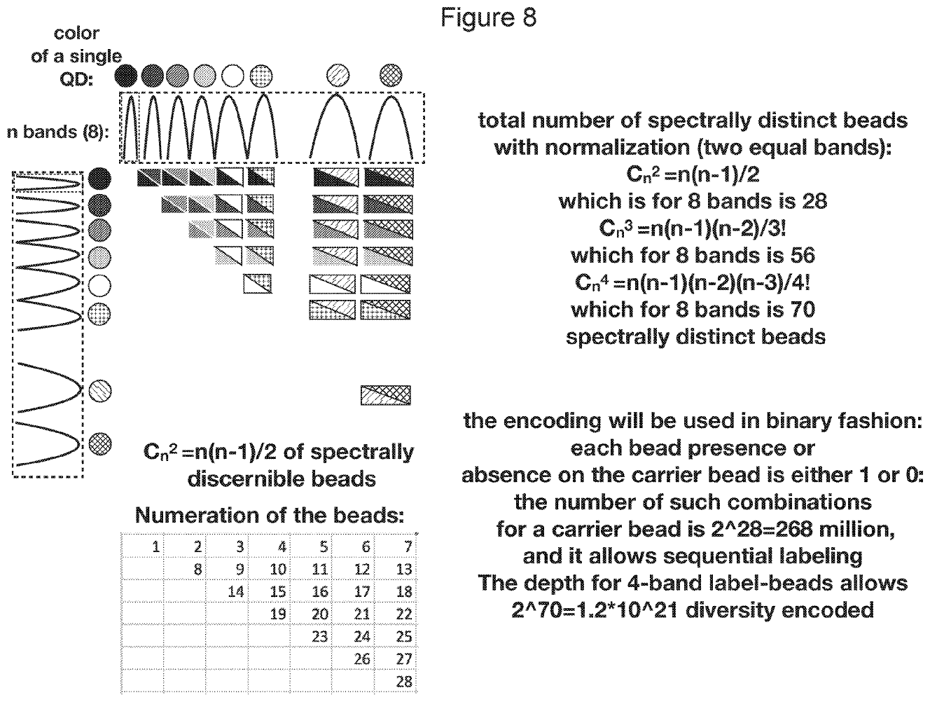

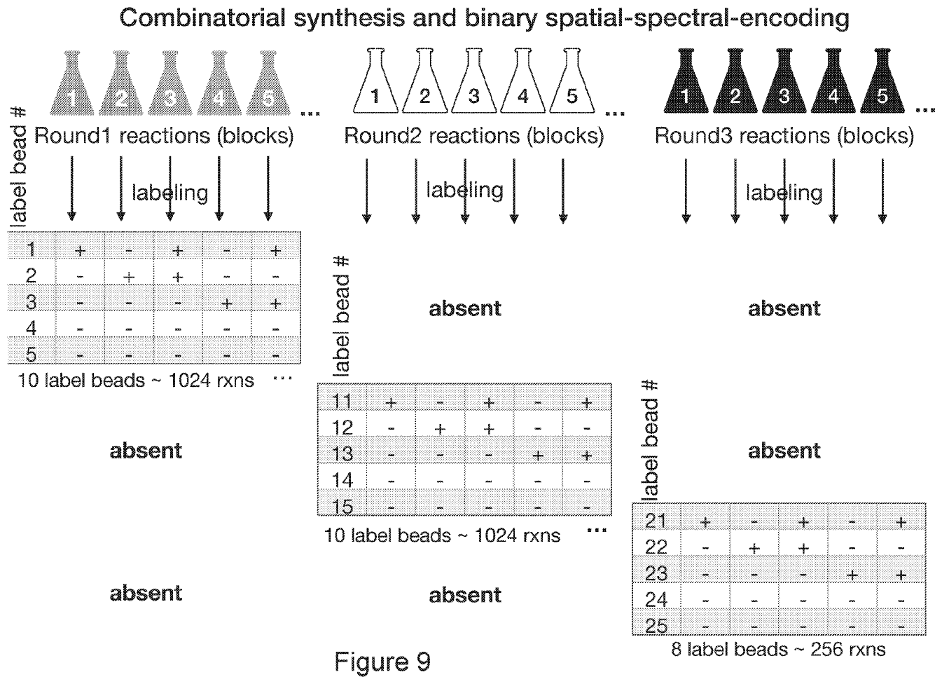

Referring to FIGS. 7-9, a system for binary encoding of the carrier-beads with the label-beads is described. Referring to FIG. 7, exemplary fluorescent spectra for a plurality of unique fluorescent labels are shown. The system for binary encoding of carrier-beads is based on a relatively small number N of different types of label-beads as described herein. The label beads are used to prepare an exponentially large number, 2^{N}, of carrier-beads with unique fluorescent encodings that are identifiable and distinguishable using the optical interrogation techniques described above. The presence of the absence of label-beads of a given type on the surface or in the bulk of a carrier-bead provides 1 bit of encoding for the carrier-bead. All possible combinations of beads of N different types being present or absent on the surface or in the bulk of the carrier-bead provide N bits of encoding and 2^{N} unique fluorescent encodings. The availability of, e.g., 10, 20, and 30 different types of label-beads makes it possible to prepare carrier-beads with, respectively, 2¹⁰≈10³, 2²⁰≈10⁶, and 2³⁰≈10⁹ unique fluorescent encodings. FIG. 8 illustrates the binary encoding system, according to the present invention.

According to the system for binary encoding of carrier-beads, when label-beads of a certain type are intended to be present on the surface of the carrier-beads, the carrier-beads are mixed with the label-beads of this type in a proportion that results in an average number M of the label-beads of this type attached to the surface of each carrier-bead. The number M is sufficiently large to lead to a practically negligible probability for the carrier-beads to have no label-beads of this type, as expected from a random Poissonian process, e.g., probabilities of 0.7%, 0.25%, 0.1%, 0.035%, and 0.012% for M=5, 6, 7, 8, and 9, respectively.

According to the system for binary encoding of carrier-beads, as described herein, the highest practically probable total number of the label-beads on the surface of a carrier-bead is sufficiently small and the area of the surface is sufficiently large. This ensures a practically negligible probability that the attachment of additional label-beads to the surface of the carrier-bead would be substantially impeded by the reduction of the surface area available for the attachment, as caused by the label-beads already attached to the surface.

When label-beads of a certain type are intended to be present in the bulk of the carrier-beads, the material used to produce the carrier-beads is mixed with the label-beads of this type in a proportion that results in an average number M of the label-beads of this type captured in the bulk of each carrier-bead. The number M is sufficiently large to lead to a practically negligible probability for the carrier-beads to have no label-beads of this type, as expected from a random Poissonian process, e.g. probabilities of 0.25%, 0.1%, 0.035%, and 0.012% for M=6, 7, 8, and 9, respectively.

Further, according to the system for binary encoding of carrier-beads, as described herein, when the practically possible maximal total number of the label-beads in the bulk of a carrier-bead is sufficiently small and the volume of the carrier-bead is sufficiently large. This ensures a practically negligible probability that the capture of additional label-beads in the bulk of the carrier-bead during the preparation of the carrier-bead would be substantially impeded by the reduction of the volume in the bulk available for capture of additional label-beads, as caused by the label-beads already captured in the bulk.

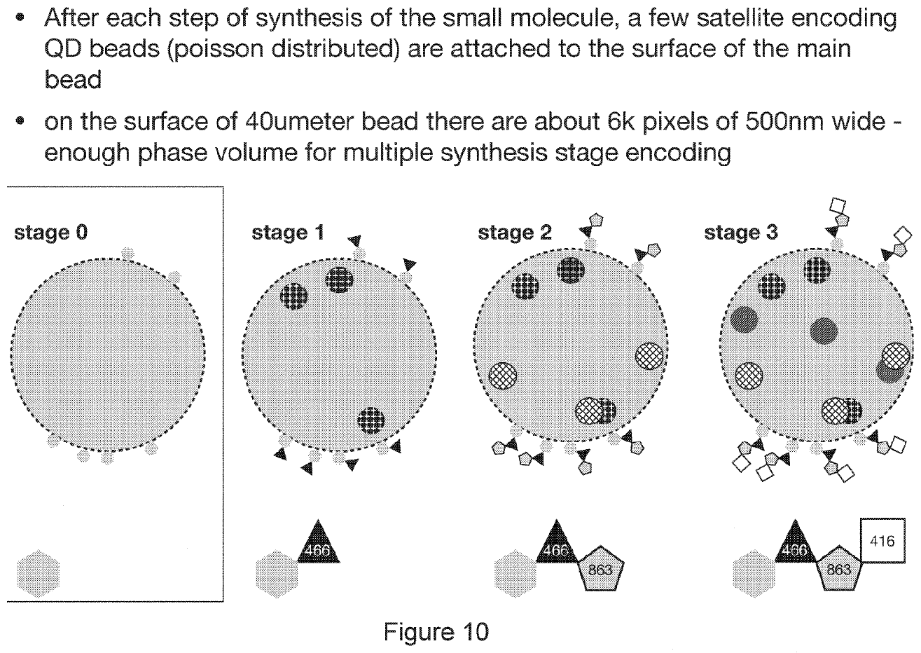

Referring now to FIGS. 10-12, a system for preparing a combinatorial library with fluorescent encoding in a combinatorial synthesis is provided. As shown in FIG. 10, after each step in the synthesis of the molecule (small or large), label-beads are attached to the surface of the carrier-bead. For example, if the 3D resolution of the optical interrogation system is l (and average between resolutions along the x, y, and z—axes), this system can resolve 4πR²/l² individual voxels at the surface of a carrier-bead with radius R. For a resolution l=1 um and carrier bead diameter 2R=40 um, the number of resolvable voxels is ˜5,000.

According to the system, first a set of label-beads is provided. The set comprises a plurality of types of label-beads, wherein all label-beads of a given type have a unique fluorescent label, as described herein. A plurality of carrier-beads, as described herein is then provided. The molecular building blocks for each step in the combinatorial synthesis are provided. A sequence of steps of combinatorial synthesis on the carrier-beads is then performed. A new molecular building block is added to each carrier-bead at each step, to form a combinatorial library with a plurality of groups of carrier-beads, each group of carrier-beads in the library having a unique synthetic compound. Each type of the new molecular building blocks bound to carrier-beads in a given step of the combinatorial synthesis is matched with a unique type of the label-beads newly attached to the surface of the carrier-beads at this step, thereby forming a population of carrier-beads with a plurality of groups, carrier-beads of each group in the population carrying a unique synthetic molecule and having a matching unique fluorescent encoding produced by a plurality of label-beads of different types with unique fluorescent labels attached to the surface of the carrier-beads in consecutive steps of the combinatorial synthesis.

The sizes of the carrier-beads and label-beads, the average numbers of the label-beads of each type attached to a single carrier-bead, and the intensities and spectra of fluorescence of different types of the label-beads are selected in a way that enables reliable and robust optical interrogation, as described herein, making it possible to identify all types of label-beads attached to each carrier-bead, and identify the unique synthetic molecule on each carrier-bead.

In the system described above each type of label-beads is used at not more than one step of the combinatorial synthesis.

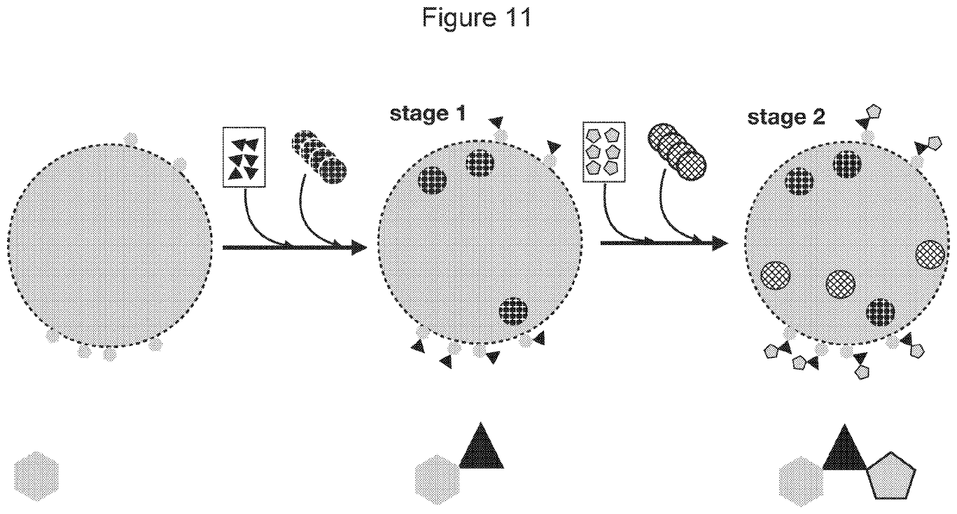

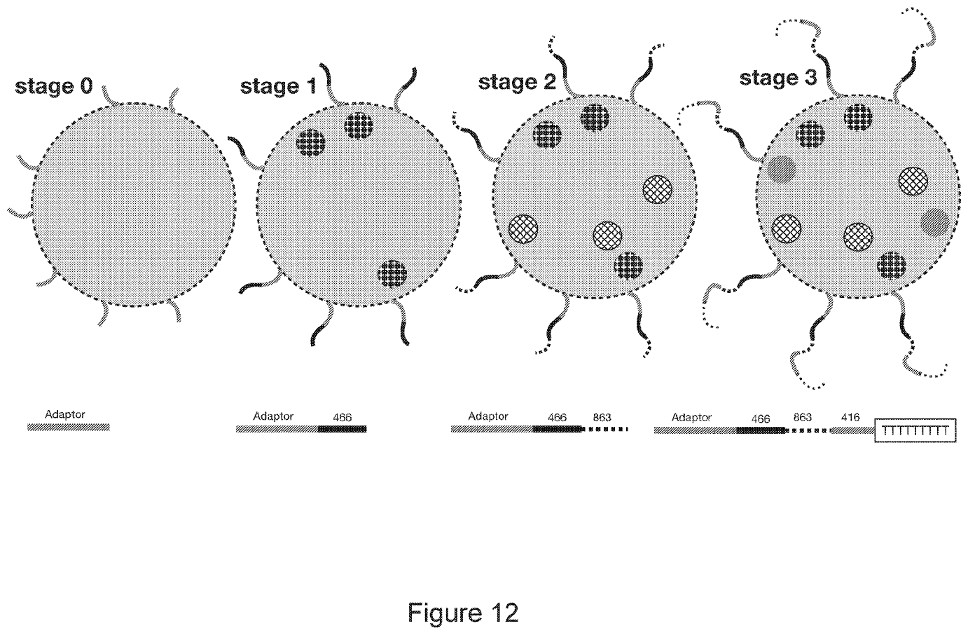

As shown in FIGS. 10, 11, and 12, the encoding system described herein can be used for a synthesis of small molecules (FIGS. 10-11) or synthesis of larger molecules (FIG. 12), e.g., DNA, peptides, etc.

As shown in FIG. 10, after each step of synthesis, a few satellite encoding QD beads, also referred to as labelling bead types, where each labelling bead type has a unique fluorescent label, (poisson distributed) are attached to the surface of the carrier bead. On the surface of 40 umeter bead there may be about 6 k pixels of 500 nm wide, enough phase volume for multiple synthesis stage encoding.

Quantum dots and beads doped with QD are practically inert for organic synthesis applications which allows any chemistry for chemical library construction. Chemical library carrier-beads could be functionalized with RNA capturing barcoded DNA oligos with combinatorial barcodes after releasable chemical library is complete, which will enable measurement of transcriptional (epigenetic, etc.) response using NGS, which could be traced back to the carrier-bead by sequencing DNA barcode in-situ and decoded by the same optical interrogation of the label-beads: linking cellular-transcriptional phenotype with a hit from DNA-non-compatible chem-library. The encoding capacity reachable with the binary encoding with label-beads is beyond cell based assays (millions of cells) and close to biochemical assays (10Λ12Λ-10Λ16 members).

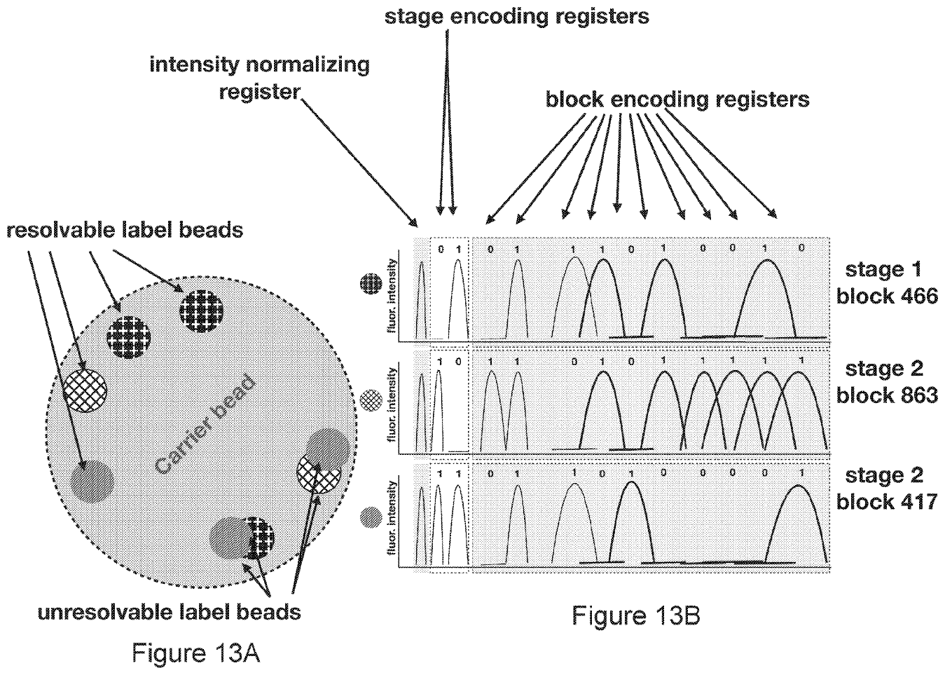

Referring to FIG. 13, the information coding capacity depends on how many chemical library synthesis stages are used.

According to the present invention, a combinatorial library based on a population of fluorescently encoded carrier-beads is provided. Each carrier-bead has a synthetic molecule comprised of a certain number of molecular building blocks added at consecutive steps of the combinatorial synthesis and a plurality of label-beads of the same number of different types attached to the surface of the carrier-bead at consecutive steps of the combinatorial synthesis. Each carrier bead also has a unique combination of label-beads to identify the synthetic molecule on the carrier-bead. Each label-bead has a unique fluorescent label which is optically resolvable from the unique fluorescent labels of other label-beads of all other types on the surface of the carrier-bead.

Referring again to FIG. 11, the type of label-beads attached to a carrier-bead at a given step of the combinatorial synthesis uniquely matches the type of the molecular building block added to the carrier-bead at the same step of the combinatorial synthesis, such that the set of types of the label-beads attached to a given carrier-bead uniquely encodes the set of the molecular building blocks in the synthetic molecules on the carrier-bead.

All carrier-beads belonging to a given group in the population of the carrier-beads carry the same synthetic molecules comprised of the same sequence of molecular building blocks and have the same fluorescent encoding, whereas beads belonging to different groups carry different synthetic molecules and have different fluorescent encodings that are optically resolvable on the carrier-beads with the optical interrogation techniques described herein.

In a combinatorial library produced by the systems and methods described herein, each type of label-bead is used at not more than one step of the combinatorial synthesis, such that the fluorescent encoding uniquely defines both the types of the building blocks in the synthetic molecules and he order in which these blocks were added.

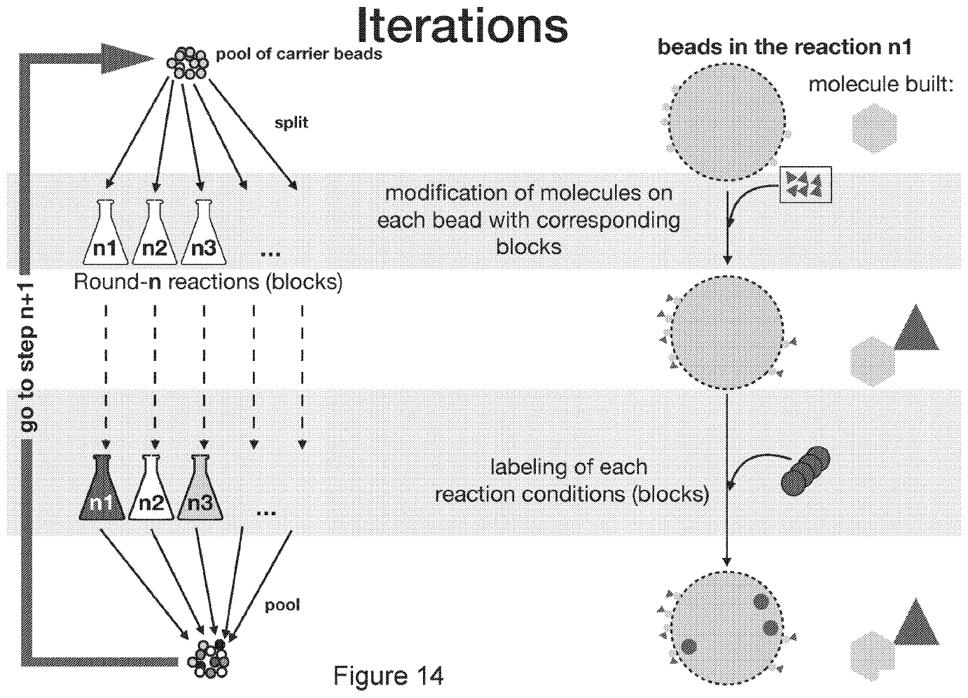

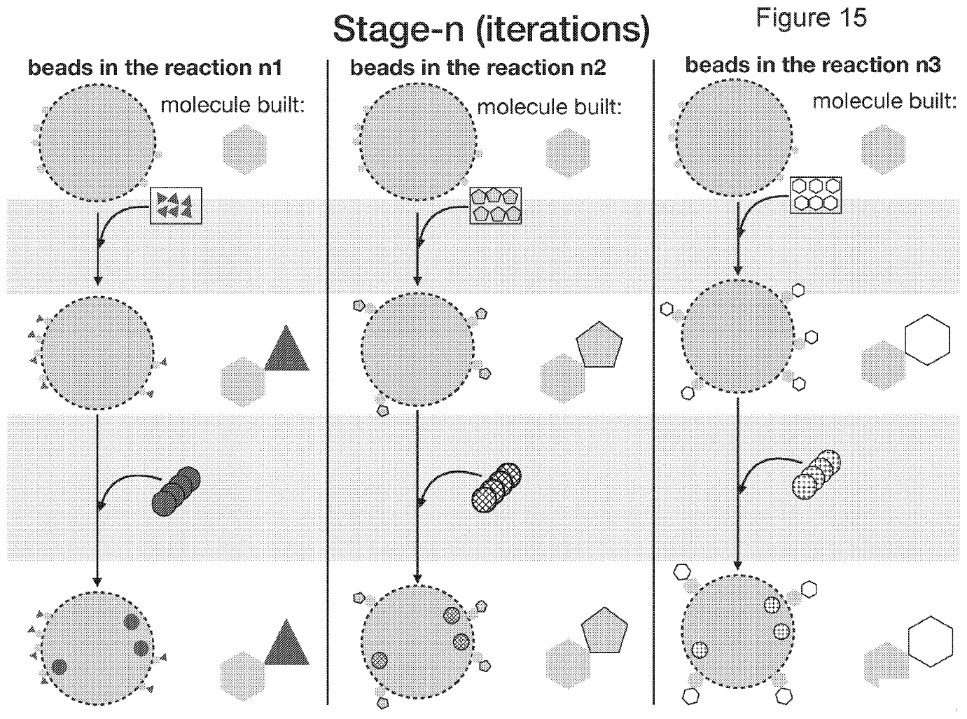

Referring now to FIG. 14 and FIG. 15, according to another embodiment, a method of labelling carrier-beads in a solid phase combinatorial synthesis is provided. According to the method, a carrier-bead having a primary compound attached thereto is provided. A set of molecular building blocks for each step of the synthesis is also provided. The carrier-bead having the primary compound is combined with a molecular building block to form synthetic molecule 1 that is comprised of the primary compound and the added building block. The carrier-bead with synthetic molecule 1 is attached to a plurality of label-beads of the type matching the type of the newly added molecular building block. The above steps are repeated and the molecular synthesis is accomplished by adding new molecular building blocks to the synthetic molecules on the surface of the carrier-bead and attaching label-beads of the matching type to the surface of the carrier-bead to form carrier-beads with synthetic molecules 2, 3, 4, . . . having, respectively, 2, 3, 4, . . . molecular building blocks and with label-beads of, respectively, 2, 3, 4, . . . types matching the molecular building blocks attached to the surface of the carrier-bead.

A synthetic end product is obtained and each group of carrier-beads has a corresponding unique set of labelling beads to identify the synthetic end product. The label-beads with a unique set of types of label-beads on each carrier bead is compatible with the optical interrogation techniques described herein, making it possible to reliably detect the types of label-beads attached to the surface of each carrier-bead and identify the synthetic end product.

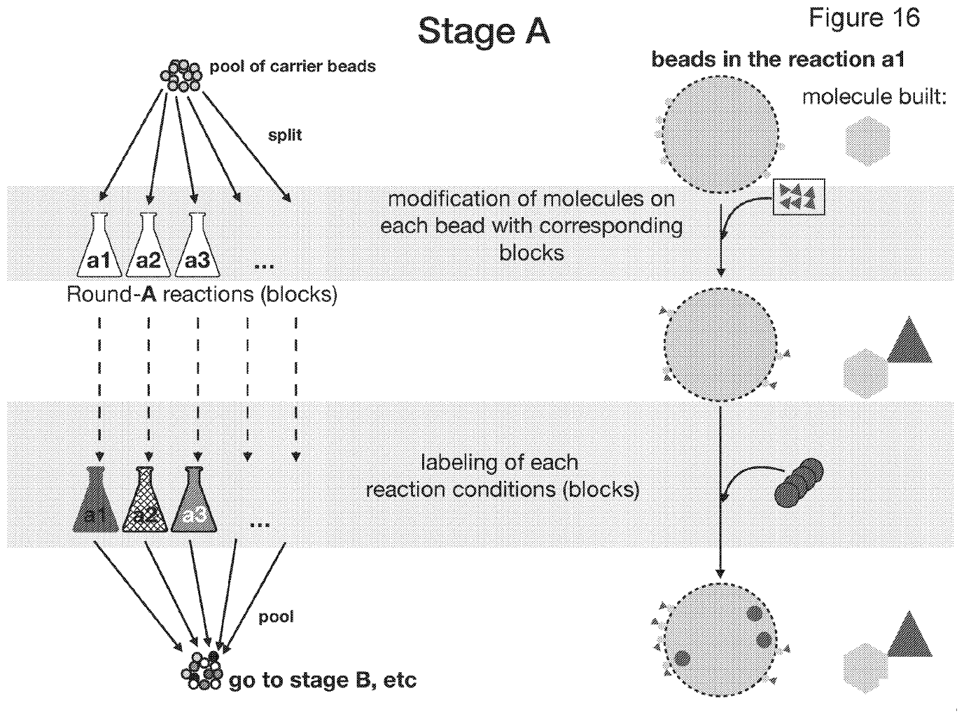

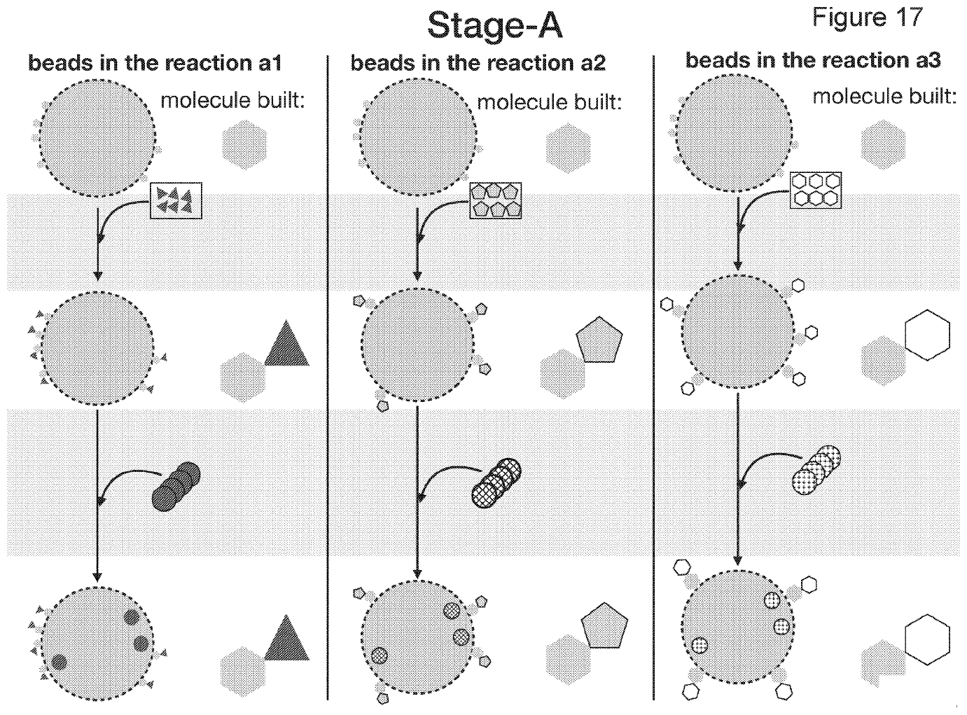

Referring now to FIGS. 16-24, a multi-stage combinatorial synthesis is shown. According to the invention, a system for preparing a combinatorial library with binary fluorescent encoding in a combinatorial synthesis is shown. The system comprises selecting sets of molecular building blocks used at each of the steps of the combinatorial synthesis. The number of different building blocks in the sets is calculated. For example the number of different building blocks used in each phase A, B, C, a1, a2, a3, etc. as shown in FIGS. 17-24 is calculated. . . . , B1, B2, B3 . . . , respectively.

A set of label-beads of a plurality of types is provided, wherein all label-beads of a given type have a unique fluorescent label, as described herein. The different types of label-beads are split into cohorts 1, 2, 3, . . . with the number of cohorts equal to or greater than the number of steps of the combinatorial synthesis, and with the number of types of the label-beads in cohorts 1, 2, 3, . . . , N1, N2, N3, respectively, satisfying the equations N1≥log₂(B1), N2≥log₂(B2), N3≥log₂(B3) . . . , such that the molecular building blocks added at each step of the combinatorial synthesis can be binary encoded by the label-beads from the corresponding cohort of types, as described herein.

A set of one or a plurality of carrier-beads, as described herein is then provided. A sequence of steps of combinatorial synthesis is performed on each of the carrier-beads, with a new molecular building block added to each bead at each step, to form a combinatorial library with a plurality of groups of carrier-beads, each group of carrier-beads in the library having a unique synthetic compound. In each step of the combinatorial synthesis, a unique combination of types of label-beads from the cohort matching the step of the synthesis is attached to the surface of the carrier-beads, thereby forming a population of carrier-beads, each carrier-bead having a different synthetic molecule with a plurality of groups.

The carrier-beads of each group in the population carry a unique synthetic molecule and have a matching unique fluorescent encoding produced by a plurality of label-beads of different types with unique fluorescent labels attached to the surface of the carrier-beads in consecutive steps of the combinatorial synthesis.

The sizes of the carrier and label-beads, the average numbers of the label-beads of each type attached to a single carrier-bead, and the intensities and spectra of fluorescence of different types of the label-beads are selected in a way that allows for reliable and robust optical interrogation as described herein, making it possible to identify all types of label-beads attached to each carrier-bead, and the corresponding unique synthetic molecule attached thereto.

For example, as shown in FIGS. 16 and 17, in phase A, a first set of label-beads, each having a starting molecule, is split into three reactions, a1, a2, a3, and combined with a first molecular building block, the first molecular building block being different in each of the three reactions a1, a2, a3. A unique combination of label-beads binary encoding the first molecular building block is then matched with each type of first molecular building blocks and attached to the carrier-bead.

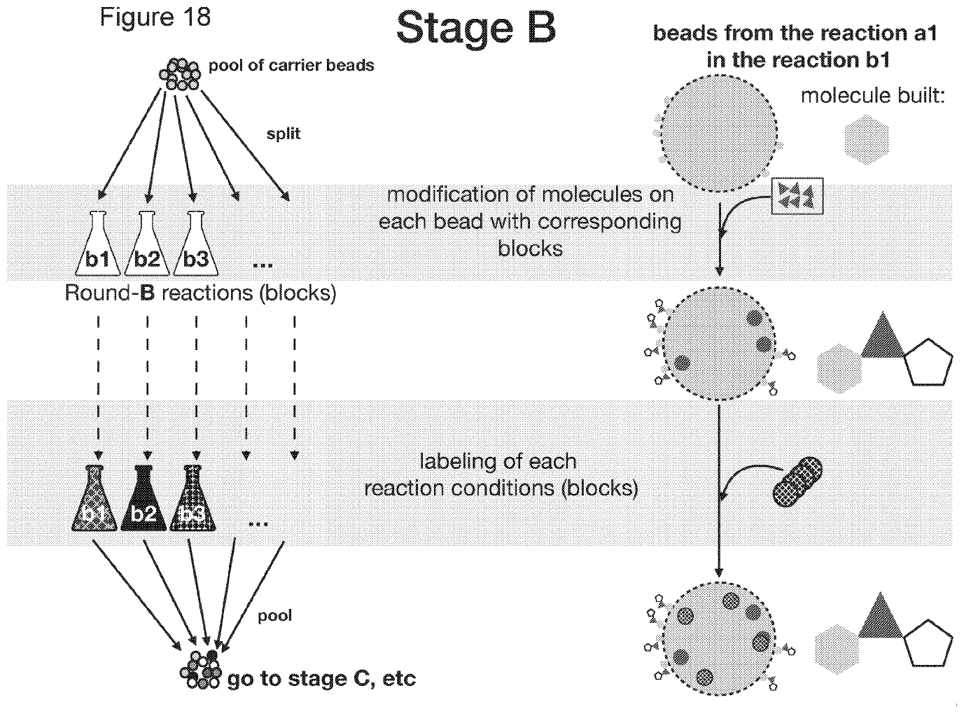

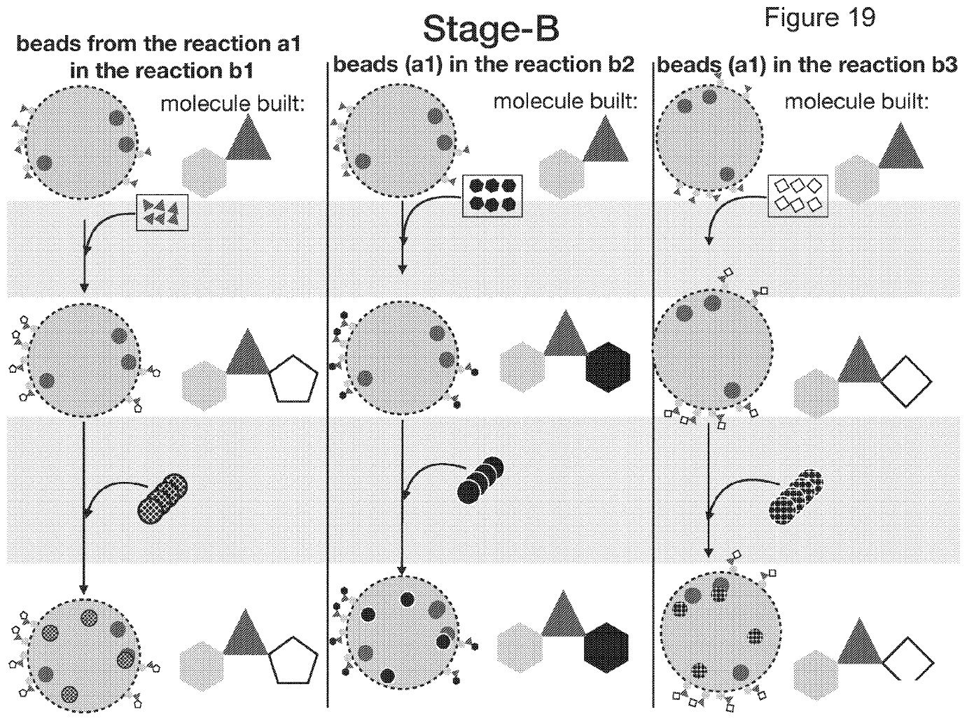

As shown in FIGS. 18 and 19, in Stage B, next, the carrier-beads having reactions a1, a2, and a3, and their corresponding bead-labels are combined and split again into reactions b1, b2, and b3. Reactions b1, b2, and b3 are combined with a second molecular building block, the second molecular building block being different in each of the three reactions b1, b2, b3. A uniquely unique combination of label-beads binary encoding the second molecular building block is then matched with each type of second molecular building blocks and attached to the carrier-bead in each of the reactions b1, b2 and b3.

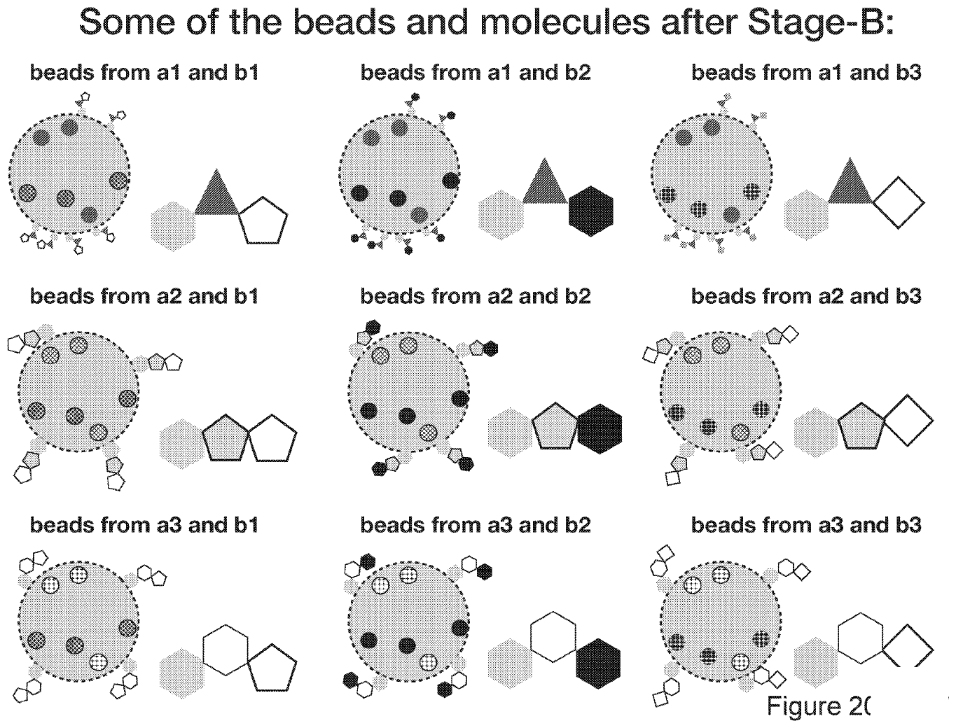

FIG. 20 shows the combination of beads and unique fluorescent labels after Stage B of the combinatorial synthesis.

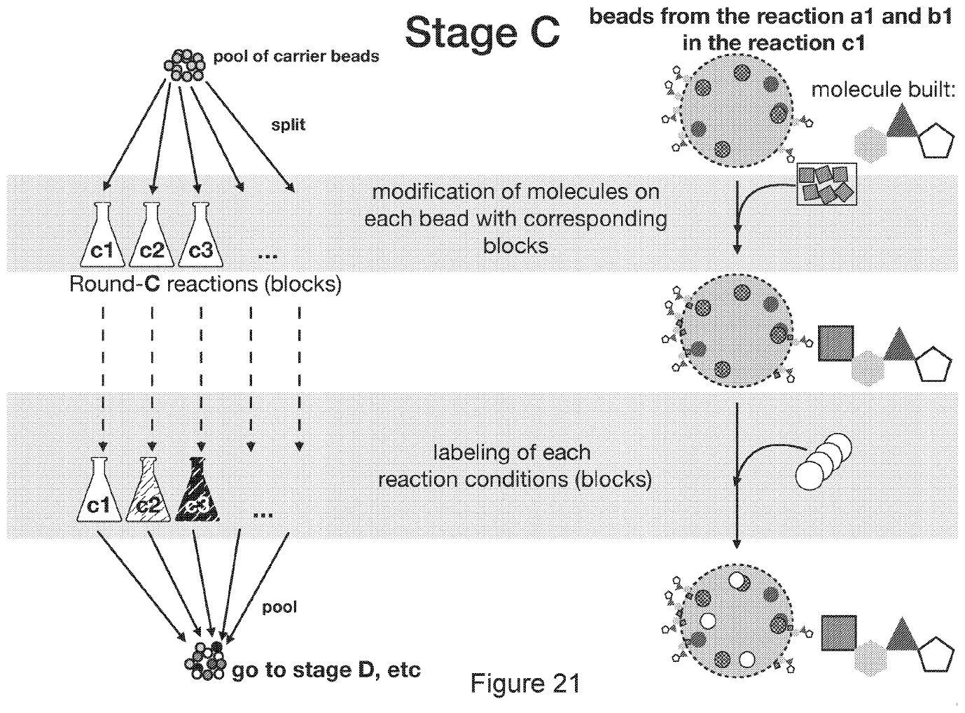

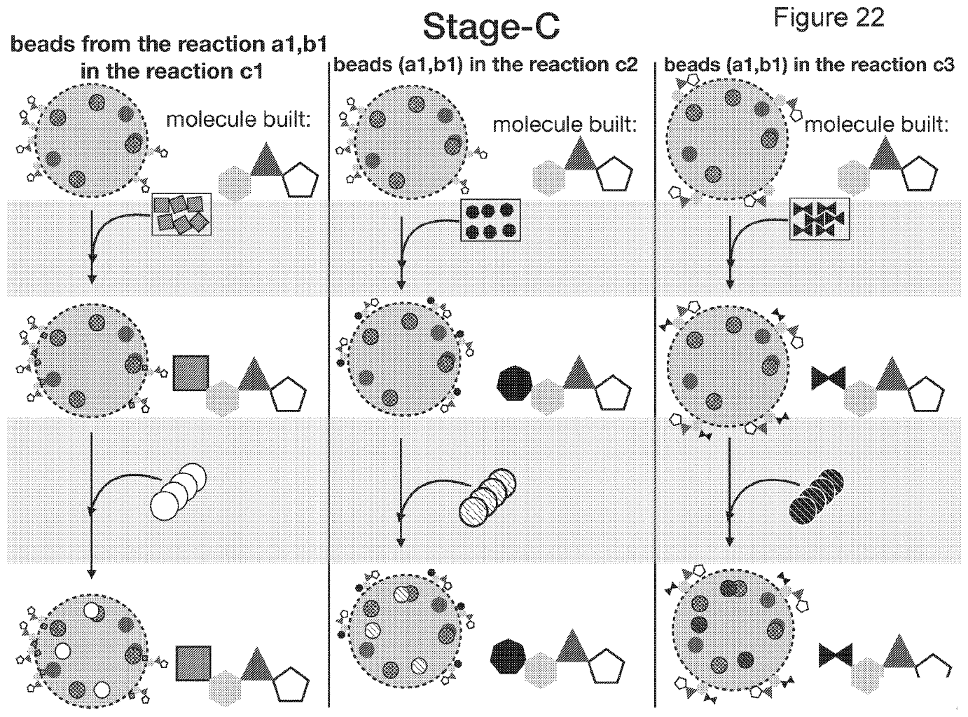

As shown in FIGS. 21 and 22, in Stage C, next, the carrier-beads having the product of reactions b1, b2, and b3, and their corresponding bead-labels are combined and split again into reactions c1, c2, and c3. Reactions c1, c2, and c3 are combined with a third molecular building block, the third molecular building block being different in each of the three reactions c1, c2, c3. A unique combination of label-beads binary encoding the third molecular building block is then matched with each type of third molecular building block and attached to the carrier-bead in each of the reactions c1, c2 and c3.

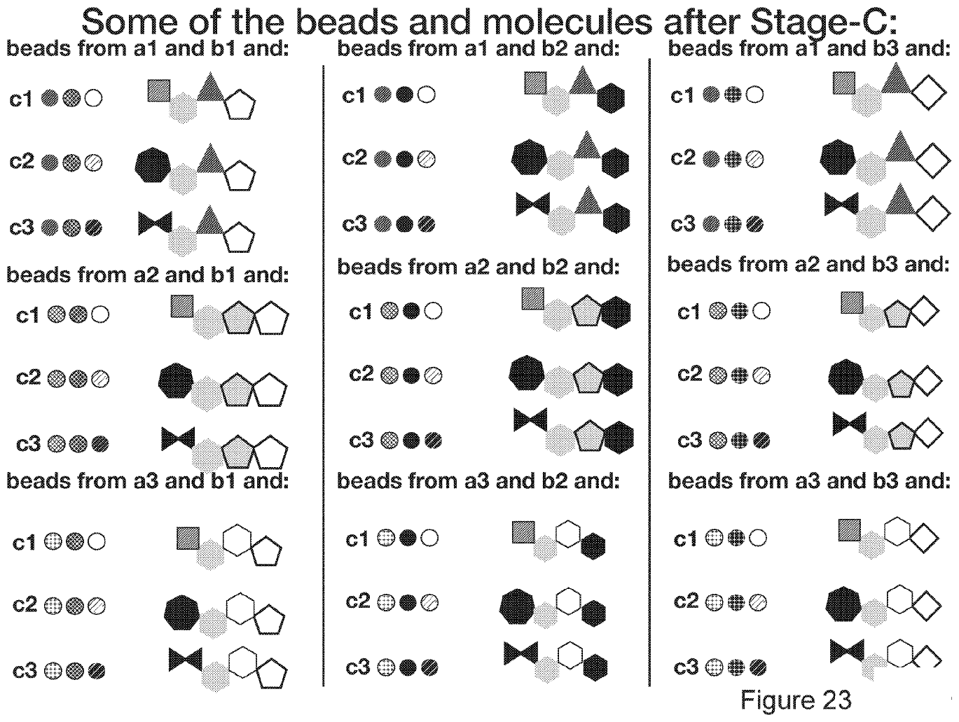

FIG. 23 shows the combination of beads and labels after Stage C of the combinatorial synthesis. After completion of the synthesis, the carrier-beads of each group in the population carry a unique synthetic molecule and have a matching unique fluorescent encoding produced by a plurality of label-beads of different types, each with unique fluorescent labels, attached to the surface of the carrier-beads in consecutive steps of the combinatorial synthesis.

Accordingly, a combinatorial library based on a population of carrier-beads, each bead having a unique fluorescent encoding as described herein is also provided. Each carrier-bead in the population carries a synthetic molecule comprised of a certain number of molecular building blocks added at consecutive steps in a combinatorial synthesis. The types of label-beads are split into cohorts 1, 2, 3, . . . , with the number of cohorts being equal to or greater than the number of steps in the combinatorial synthesis. The numbers of label-beads in cohorts used for steps 1, 2, 3, . . . , N1, N2, N3, respectively, satisfy the equations N1≥log₂(B1), N2≥log₂(B2), N3≥log₂(B3) . . . , such that the molecular building blocks added at each step of the combinatorial synthesis are encoded by the label-beads, as described herein, from the corresponding cohort of types of label-beads, as also described herein.

The combination of types of label-beads attached to a carrier-bead at each step of the combinatorial synthesis uniquely matches the type of the molecular building block newly added to this carrier-bead at the same step of the combinatorial synthesis, such that the set of all types of the label-beads attached to each carrier-bead uniquely encodes the set of the molecular building blocks in the synthetic molecules on the carrier-bead. In addition, all carrier-beads belonging to a given group in the population of the carrier-beads carry the same synthetic molecules comprised of the same sequence of molecular building blocks and have the same fluorescent encoding, whereas beads belonging to different groups carry different synthetic molecules and have different fluorescent encodings that are optically resolvable with the optical interrogation techniques as described herein.

According to one embodiment, one or more of the synthetic molecules attached to the carrier-beads in the combinatorial library described herein can optionally be cleavable by light or chemically from the carrier-bead. In another embodiment, each carrier-bead can be chemically or physically disintegrated, while the integrity of the label-beads attached to the carrier-beads is preserved.

In each of the embodiments described herein, the synthetic molecules on the carrier-beads can be small molecules, large molecules or macromolecules.

A method of solid phase synthesis on carrier-beads is also provided. The method comprises providing a population of carrier-beads having a primary compound attached thereto. The label beads of different types, according to the present invention, are split into a number of cohorts equal to or greater than the number of the steps of synthesis and with the numbers of beads in cohorts used for synthesis steps 1, 2, 3, . . . , N1, N2, N3, respectively, satisfying the equations N1≥log₂(B1), N2≥log₂(B2), N3≥log₂(B3), where B1, B2, B3 . . . , are the numbers of different building blocks used in steps, 1, 2, 3, . . . respectively, such that the molecular building blocks added at each step of the combinatorial synthesis can be binary encoded by the label-beads from the corresponding cohort of types, as described herein. Carrier-beads having the primary compound are combined with a molecular building block to form synthetic molecule 1 that is comprised of the primary compound and the added building block. A plurality of label-beads from cohort 1 with the combination of types matching the type of the newly added molecular building block are attached to the carrier-bead with synthetic molecule 1. The synthesis is repeated by adding a new molecular building blocks to the synthetic molecules on the surface of the carrier-bead and attaching label-beads of the matching combination of types to the surface of the carrier-bead at each step of combinatorial synthesis to form carrier-beads with synthetic molecules 2, 3, 4, . . . having, respectively, 2, 3, 4, . . . molecular building blocks and with label-beads from the cohorts, 2, 3, 4, . . . , respectively, and with the combination of labelling bead types matching the types of the molecular building blocks attached to the surface of the carrier-bead at the corresponding steps of the synthesis.

An end product is obtained, having carrier-beads compatible with the optical interrogation techniques described herein, making it possible to reliably detect all the types of label-beads attached to the surface of each carrier-bead.

According to another embodiment, a method of decoding the composition of the synthetic molecules on the carrier-beads according to the present invention is described herein. The method comprises first providing a population of carrier-beads comprised of a plurality of groups, each group of the carrier-beads in the population having a unique synthetic compound on the surface and a unique fluorescent encoding provided by a unique set of types of label-beads attached to the surface of the carrier-beads. The carrier beads are each optically interrogated with the optical interrogation techniques described herein. The results of the optical interrogation are used to identify all individual types of the label-beads on the surface of each carrier-bead of interest. The newly identified types of the label-beads are matched with the types of molecular building blocks to identify all individual molecular building blocks in the synthetic molecules on the carrier-bead and the order in which these building blocks were added. The information on the type and order of the newly identified building blocks is used to obtain the structure of the synthetic molecules on the carrier-bead.

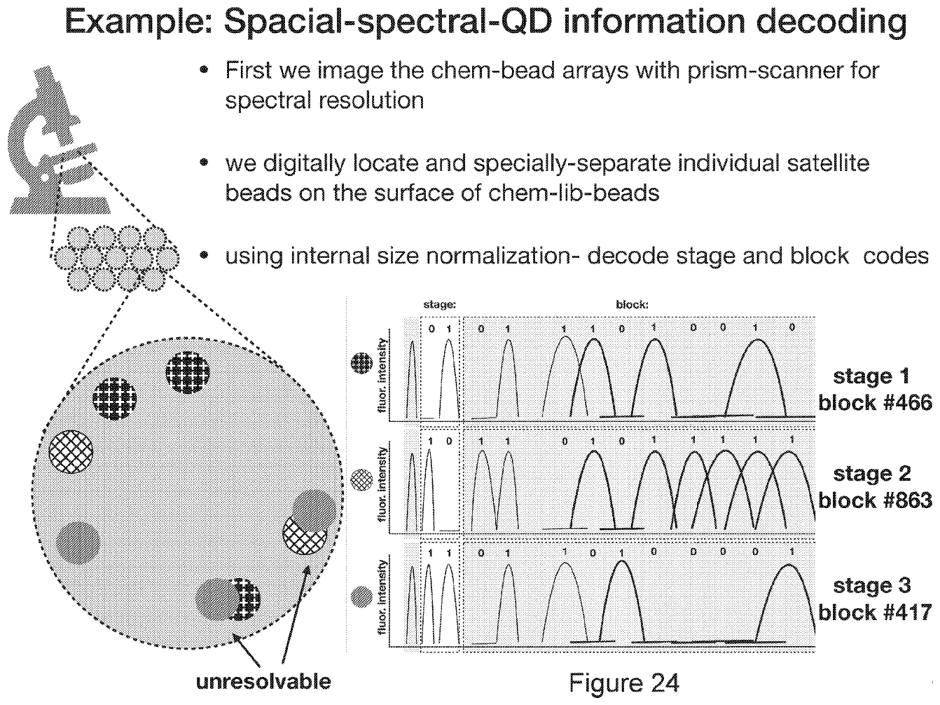

Referring now to FIG. 24, an example of special-spectral quantum dot information decoding is shown schematically.

According to another embodiment, a method of screening a chemical library for molecules and macromolecules having certain desired properties is provided. According to the method, first, a population of carrier-beads with a fluorescently encoded library of molecules or macromolecules as described herein is provided. The population of carrier-beads is contacted with assay reagents to assay the molecules or macromolecules on the population of carrier beads for a certain desired outcome.

The population of carrier-beads is then optically interrogated as described herein and the carrier-beads for which the desired outcomes were observed are identified. The results of the optical interrogation are then used to identify the synthetic structure of the individual molecules or macromolecules on the carrier-beads for which the desired assay outcomes were observed.

The assay may be performed in microwells, aqueous droplets, hydrogels, or living tissues.

The outcomes of the assay can be observed using readouts which are biochemical, lysate based, or cell based. The desired information can also be obtained via sequencing or counting DNA or RNA molecules.

The carrier-beads, label-beads, encoding systems and methods described herein according to the present invention may be used in a variety of systems and applications. Certain embodiments have been described herein, with reference to various compounds, materials and methods. However, it will be understood by those of skill in the art, that other compounds, materials, methods and applications for the Molecular Library Encoding Systems and Methods as described are envisioned to be within the scope of the invention and are not limited by the above-description of preferred embodiments, as will be understood by those of skill in the art, with reference to this disclosure.

What is claimed is:

1. A set of carrier beads comprising:
a set of labelling beads, the set of labelling beads comprising:
a plurality of labelling bead types, each labelling bead type being adapted to attach to the surface of a carrier bead or be captured in the bulk of a carrier bead, each labelling bead type having unique fluorescent label which is optically resolvable from the unique fluorescent label of every other labelling bead type when attached to the surface of a carrier bead or captured in the bulk of a carrier bead;
wherein:
the unique fluorescent label on each labelling bead type comprises one, two or more fluorescence emission bands and one, two or more fluorescence excitation bands, each fluorescence emission band being distinguishable by one, two or more of: (i) the representative wavelength of emission, (ii) the intensity of emission, (iii) the characteristic wavelength of the excitation band exciting the emission, and (iv) the efficiency of excitation in this excitation band exciting the emission; and
the unique fluorescent label on each labelling bead type is spectrally distinct from the unique fluorescent labels on the other labelling bead types in the set of labelling beads, such that the plurality of labelling bead types, in combination, are each uniquely labelled.

2. A set of carrier beads according to claim 1, wherein each labelling bead type is encoded with two or more distinguishable fluorescence emission bands, each individual band being selected from a larger set of four or more fluorescence emission bands.

3. The set of carrier beads according to claim 1, wherein the unique fluorescent label of at least one of the labelling bead types is resultant from fluorescent quantum dots.

4. The set of carrier beads according to claim 1, wherein the diameter of the carrier beads is three or more times greater than the diameter of the labelling beads.

5. The set of carrier beads according to claim 1, wherein the unique fluorescent label on at least one labelling bead type is identifiable with an optical interrogation technique, and
wherein the carrier beads are optically interrogated with three-dimensional (3D) resolution and the diameter of the smallest optically resolved 3D region is smaller than the diameter of the carrier beads.

6. The set of carrier beads according to claim 5, wherein the diameter of the smallest optically resolved 3D region is 10 or more times smaller than the diameter of the carrier beads.

7. The set of carrier beads according to claim 5, wherein the optical interrogation technique distinguishes between the spectra of fluorescence of the individual labelling bead types, comprising the wavelengths and intensity of fluorescence, when the labelling beads are attached to the surface or captured in the bulk of the carrier beads.

8. The set of carrier beads according claim 5, wherein the optical interrogation technique is confocal fluorescence imaging with spectral resolution or confocal fluorescence spectroscopy.

9. The set of carrier beads according to claim 5, wherein the wavelength and intensity sensitivities and the 3D spatial resolution are sufficiently high and the characteristic distance between the labeling beads is sufficiently large, to reliably detect the type of an absolute majority of the individual labelling beads attached to the surface or captured in the bulk of the carrier bead, as to ensure that the proportion of labelling beads that are not accounted for or whose type is not identified or misidentified is practically negligible.

10 The set of carrier beads according to claim 9, wherein the diameter of the smallest resolved 3D region is smaller than the diameter of the labelling beads.

11. The set of carrier beads according to claim 9, wherein the diameter of the smallest resolved 3D region is smaller than average distance between the labelling beads.

12. A binary encoding system for identification of carrier beads, the system comprising:
providing a number N of different labelling bead types, the N different labelling bead types being used to prepare an exponentially large number, 2^{N}, of carrier bead types, each labelling bead type being adapted to attach to the surface of a carrier bead or be captured in the bulk of a carrier bead, each labelling bead type having unique fluorescent label which is optically resolvable from the unique fluorescent label of other labelling bead type when attached to the surface of a carrier bead or captured in the bulk of a carrier bead;
wherein:
the unique fluorescent label on each labelling bead type comprises one, two or more fluorescence emission bands and one, two or more fluorescence excitation bands, each fluorescence emission band being distinguishable by one, two or more of: (i) the representative wavelength of emission, (ii) the intensity of emission, (iii) the characteristic wavelength of the excitation band exciting the emission, and (iv) the efficiency of excitation in this excitation band exciting the emission; and
the unique fluorescent label on each labelling bead type is spectrally distinct from the unique fluorescent label on the other labelling bead types in the set of labelling beads, such that the plurality of labelling bead types, in combination, are each uniquely labelled, and the unique fluorescent label on each labelling bead type is identifiable with an optical interrogation technique; and
optically interrogating the carrier beads with three-dimensional (3D) resolution and the diameter of the smallest optically resolved 3D region is smaller than the distance between the carrier beads, wherein
the presence or the absence of a labelling bead type on the surface or in the bulk of the carrier bead provides 1 bit of encoding for the carrier bead type, and
wherein all possible combinations of beads of N different types being present or absent on the surface or in the bulk of the carrier bead provide N bits of encoding and 2^{N} unique fluorescent encodings, and
wherein the availability of 10, 20, and 30 different types of labelling beads makes it possible to prepare carrier beads with, respectively, 2¹⁰≈10₃, 2₂₀≈10⁶, and 2³⁰≈10⁹ unique fluorescent encodings.

13. The system for binary encoding of carrier beads according to claim 12, wherein, when labelling beads of a certain labelling bead type are intended to be attached to the surface of the carrier beads, the carrier beads are mixed with the labelling beads of this type in a proportion that results in an average number M of the labelling beads of this type attached to the surface of each carrier bead, and wherein the number M is sufficiently large to lead to a practically negligible probability for the carrier beads to have no labelling beads of this type, as expected from a random Poissonian process, having probabilities of 0.25%, 0.1%, 0.035%, and 0.012% for M=6, 7, 8, and 9, respectively.

14. The system for binary encoding of carrier beads according to claim 12, wherein the highest practically probable total number of the labelling beads on the surface of a carrier bead is sufficiently small and the area of the surface is sufficiently large, as to ensure a practically negligible probability that the attachment of additional labelling beads to the surface of the carrier bead would be substantially impeded by the reduction of the surface area available for the attachment, as caused by the labelling beads already attached to the surface.

15. The system for binary encoding of carrier beads according to claim 14, wherein the highest practically probable total number of the labelling beads on the surface of a carrier bead is sufficiently small and the area of the surface is sufficiently large, as to ensure that the average distance between the beads is greater than the diameter of the smallest optically resolved 3D region.

16. The system for binary encoding of carrier beads according to claim 15, wherein the average distance between the beads is >10 times greater than the diameter of the smallest optically resolved 3D region.

17. The system for binary encoding of carrier beads according to claim 12, wherein, when labelling beads of a certain labelling bead type are intended to be present in the bulk of the carrier beads, the material used to produce the carrier beads is mixed with the labelling beads of this type in a proportion that results in an average number M of the labelling beads of this type captured in the bulk of each carrier bead, wherein the number M is sufficiently large to lead to a practically negligible probability for the carrier beads to have zero labelling beads of this type, as expected from a random Poissonian process, having probabilities of 0.25%, 0.1%, 0.035%, and 0.012% for M=6, 7, 8, and 9, respectively.

18. The system for binary encoding of carrier beads according to claim 12, wherein the practically possible maximal total number of the labelling beads in the bulk of a carrier bead is sufficiently small and the volume of the carrier bead is sufficiently large, as to ensure a practically negligible probability that the capture of additional labelling beads in the bulk of the carrier bead during the preparation of the carrier bead would be substantially impeded by the reduction of the volume in the bulk available for capture of additional labelling beads, as caused by the labelling beads already captured in the bulk.

19. The system for binary encoding of carrier beads according to claim 12, wherein the practically possible maximal total number of the labelling beads in the bulk of a carrier bead is sufficiently small and the volume of the carrier bead is sufficiently large, as to ensure that the average distance between the beads is greater than the diameter of the smallest optically resolved 3D region.

20. The system for binary encoding of carrier beads according to claim 15, wherein the average distance between the beads is >5 times greater than the diameter of the smallest optically resolved 3D region.

21. A system for preparing a combinatorial library with fluorescent encoding in a combinatorial synthesis, the system comprising:
a) providing a set of labelling beads comprised of a plurality of labelling bead types, each labelling bead type being adapted to attach to the surface of a carrier bead or be captured in the bulk of a carrier bead, each labelling bead type having unique fluorescent label which is optically resolvable from the unique fluorescent label of other labelling bead type when attached to the surface of a carrier bead or captured in the bulk of a carrier bead;
wherein:
the unique fluorescent label on each labelling bead type comprises one, two or more fluorescence emission bands and one, two or more fluorescence excitation bands, each fluorescence emission band being distinguishable by one, two or more of: (i) the representative wavelength of emission, (ii) the intensity of emission, (iii) the characteristic wavelength of the excitation band exciting the emission, and (iv) the efficiency of excitation in this excitation band exciting the emission; and
the unique fluorescent label on each labelling bead type is spectrally distinct from the unique fluorescent label on the other labelling bead types in the set of labelling beads, such that the plurality of labelling bead types, in combination, are each uniquely labelled;
b) providing a plurality of carrier beads;
c) providing a set of molecular building blocks for a combinatorial synthesis, the combinatorial synthesis having a plurality of steps, and each step of the combinatorial synthesis having a molecular building block, each building block being the same or different;
d) performing a sequence of steps of the combinatorial synthesis on a group of carrier beads, the sequence of steps comprising adding a new molecular building block to a group of carrier beads at each step of the combinatorial synthesis to form the combinatorial library, each group of carrier beads in the library having a unique synthetic compound;
e) matching each molecular building block bound to the group carrier beads in a given sequence step of the combinatorial synthesis with a labelling bead type, thereby forming a population of different carrier bead groups, each carrier bead group having a unique synthetic molecule and corresponding unique fluorescent label encoded on the carrier bead, the unique fluorescent label being produced by the plurality of different labelling bead types, each with unique fluorescent labels, attached to the surface of the carrier beads in consecutive steps of the combinatorial synthesis;
f) identifying the labelling bead types attached to each carrier bead by an optical interrogation technique, wherein the unique fluorescent label on at least one labelling bead type is identifiable with an optical interrogation technique, and wherein the carrier beads are optically interrogated with three-dimensional (3D) resolution and the diameter of the smallest optically resolved 3D region is smaller than the diameter of the carrier beads.

22. The system of claim 21, wherein each labelling bead type is used at not more than one step of the combinatorial synthesis.

23. A combinatorial library established on a population of fluorescently encoded carrier beads, the fluorescently encoded carrier beads comprising:
a plurality of groups of carrier beads, each group of carrier beads comprising:
a synthetic molecule, the synthetic molecule having a plurality of molecular building blocks added at N consecutive steps in a combinatorial synthesis; and
a plurality of different labelling bead types attached to the surface of the carrier bead
wherein each labelling bead type has a unique fluorescent label which is optically resolvable from the unique fluorescent label of other labelling bead types on the surface of the carrier bead, and
wherein the labelling bead type attached to the carrier bead at a given step of the combinatorial synthesis uniquely matches the type of the molecular building block added to the carrier bead at the same step of the combinatorial synthesis, such that the set of labelling bead types attached to a given carrier bead uniquely fluorescently encodes the set of the molecular building blocks in the synthetic molecules on the carrier bead, and
wherein all carrier beads belonging to a given group in the population of the carrier beads carry the same synthetic molecules comprised of the same sequence of molecular building blocks and have the same fluorescent encoding, whereas beads belonging to different groups carry different synthetic molecules and have different fluorescent encodings that are optically resolvable on the carrier beads with the optical interrogation technique wherein the unique fluorescent label on at least one labelling bead type is identifiable with the optical interrogation technique, and wherein the carrier beads are optically interrogated with three-dimensional (3D) resolution and the diameter of the smallest optically resolved 3D region is smaller than the diameter of the carrier beads.

24. The combinatorial library according to claim 23, wherein each labelling bead type is used at not more than one step of the combinatorial synthesis, such that the fluorescent encoding uniquely defines both the types of the building blocks in the synthetic molecules and the order in which these blocks were added.

25. A method of labelling carrier beads in a solid phase combinatorial synthesis, the method comprising:
a) providing a carrier bead having a primary compound attached thereto;
b) providing a set of molecular building blocks for each step of the synthesis;
c) combining the carrier bead having the primary compound with a molecular building block to form synthetic molecule M1 that is comprised of the primary compound and the first added building block;
d) attaching to the carrier bead with synthetic molecule M1 a plurality of first labelling bead types, the first labelling bead type being correlated to the first molecular building block to form a carrier bead having molecule M1 and label L1;
e) adding molecular building blocks M2-M4 to the synthetic molecule M1 on the surface of the carrier bead and attaching a second labelling bead type, L2-L4 to the surface of the carrier bead to form carrier beads having synthetic molecules M1,M2,L1,L2; M1,M3,L1,L3; and M1,M4,L1,L4;
f) adding subsequent building blocks Mx and correlating labelling bead types Lx to the molecules of step e) to form carrier beads with synthetic molecules M1,M2,Mx,L1,L2; M1,M3,Mx,L1,L3Lx; and M1,M4,Mx,L1,L4,Lx, where Mx represents the set of building blocks, and Lx represents the corresponding labelling bead type;
f) obtaining, as an end product, carrier beads compatible with an optical interrogation technique, making it possible to detect the types of labelling beads attached to the surface of each carrier bead and identify the corresponding synthetic compound.

22. A method of decoding the composition of the synthetic molecules contained on the carrier beads of claim 17, 19, or claim 21, the method comprising:
a) providing a population of carrier beads comprised of a plurality of groups, each group of the carrier beads in the population having a unique synthetic compound on the surface and a unique fluorescent encoding provided by a unique set of types of labelling beads attached to the surface of the carrier beads;
b) optically interrogating each carrier bead of interest with an optical interrogation technique;
c) using the results of the optical interrogation to identify all individual types of the labelling beads on the surface of each carrier bead of interest;
d) matching the newly identified types of the labelling beads with the types of molecular building blocks to identify all individual molecular building blocks in the synthetic molecules on the carrier bead and the order in which these building blocks were added;
e) using the information on the type and order of the newly identified building blocks to obtain the structure of the synthetic molecules on the carrier bead.

23. A system for preparing a combinatorial library with binary fluorescent encoding in a combinatorial synthesis, the system comprising:
a) selecting sets of molecular building blocks used at each of the steps in a combinatorial synthesis and calculating the numbers of different building blocks in the sets used at steps 1, 2, 3, n, as B1, B2, B3, Bn, respectively;
b) providing a set of labelling beads comprising of a plurality of labelling bead types, wherein all beads of a given labeling type have a unique fluorescent label, according to claim 1;
c) splitting the plurality of types of the labelling beads in cohorts 1, 2, 3, n, with the number of cohorts equal to or greater than the number of steps of the combinatorial synthesis, and with the number of types of the labelling beads in cohorts 1, 2, 3, n . . . , N1, N2, N3, Nn, respectively, satisfying the equations N1≥log₂(B1), N2≥log₂(B2), N3≥log₂(B3) . . . , such that the molecular building blocks added at each step of the combinatorial synthesis can be binary encoded by the labelling beads from the corresponding cohort of types;
d) providing one, or a plurality of carrier beads, according to claims 1 and 4;
e) performing a sequence of steps of combinatorial synthesis on each of the carrier beads, with a new molecular building block added to each bead at each step, to form a combinatorial library with a plurality of groups of carrier beads, each group of carrier beads in the library having a unique synthetic compound;
f) matching each type of the new molecular building blocks added to carrier beads in each step of the combinatorial synthesis with a unique combination of types of labelling beads from the cohort matching the step of the synthesis and attaching the labelling beads to the surface of the carrier beads, thereby forming a population of carrier beads with a plurality of groups,
wherein carrier beads of each group in the population carry a unique synthetic molecule and have a matching unique fluorescent encoding produced by a plurality of labelling beads of different types with unique fluorescent labels attached to the surface of the carrier beads in consecutive steps of the combinatorial synthesis;
g) identifying the labelling bead types attached to each carrier bead and using the information on the bead types to identify the type and order of the molecular building blocks on the carrier bead to obtain the structure of the synthetic molecules on the carrier bead.

24. A combinatorial library established on a population of fluorescently encoded carrier beads, the fluorescently encoded carrier beads comprising:
a plurality of groups of carrier beads, each group of carrier beads comprising:
a synthetic molecule, the synthetic molecule having a plurality of molecular building blocks added at n consecutive steps in a combinatorial synthesis, wherein the numbers of different molecular building blocks added at steps 1, 2, 3, . . . n, are B1, B2, B3 . . . Bn, respectively; and
a plurality of different labelling bead types attached to the surface of the carrier beads,
wherein each labelling bead type has a unique fluorescent label which is optically resolvable from the unique fluorescent label of other labelling bead types on the surface of the carrier bead, and
wherein the types of the labelling beads are dividing into cohorts with beads from each cohort used at not more than one step of the combinatorial synthesis,
wherein the numbers of labelling beads in cohorts used for steps 1, 2, 3, . . . n; are N1, N2, N3, Nn, respectively, satisfying the equations N1≥log₂(B1), N2≥log₂(B2), N3≥log₂(B3) . . . , such that the molecular building blocks added at each step of the combinatorial synthesis can be binary encoded by the labelling beads from the corresponding cohort of labelling bead types, according to claim 12, and
wherein the combination of types of labelling beads attached to a carrier bead at each step of the combinatorial synthesis uniquely matches the type of the molecular building block newly added to this carrier bead at the same step of the combinatorial synthesis, such that the set of all types of the labelling beads attached to each carrier bead uniquely encodes the set of the molecular building blocks in the synthetic molecules on the carrier bead, and
wherein all carrier beads belonging to a given group in the population of the carrier beads carry the same synthetic molecules comprised of the same sequence of molecular building blocks and have the same fluorescent encoding, whereas beads belonging to different groups carry different synthetic molecules and have different fluorescent encodings that are optically resolvable on the carrier beads with the optical interrogation technique wherein the unique fluorescent label on at least one labelling bead type is identifiable with the optical interrogation technique, and wherein the carrier beads are optically interrogated with three-dimensional (3D) resolution and the diameter of the smallest optically resolved 3D region is smaller than the diameter of the carrier beads.

25. A method of labelling carrier beads according to claim 12 in a solid phase synthesis, the method comprising:
a) providing a population of carrier beads having a primary compound attached thereto;
b) providing a plurality of labelling beads according to claim 12, having a plurality of different labelling bead types and splitting the labelling bead types into a number of cohorts equal to or greater than the number of the steps of synthesis and with the numbers of beads in cohorts used for synthesis steps 1, 2, 3, . . . n, as N1, N2, N3, . . .Nn, respectively, to satisfy the equations N1≥log₂(B1), N2≥log₂(B2), N3≥log₂(B3), where B1, B2, B3 . . . Bn, are the numbers of different building blocks used in steps, 1, 2, 3, . . . n, respectively, such that the molecular building blocks added at each step of the combinatorial synthesis can be binary encoded by the labelling beads from the corresponding cohort of types, according to claim 12;
c) combining the carrier bead having the primary compound with a molecular building block to form synthetic molecule 1 that is comprised of the primary compound and the added building block;
d) attaching to the carrier bead with synthetic molecule 1 a plurality of labelling beads from cohort 1 with the combination of types matching the type of the newly added molecular building block;
e) repeating b) and c) by adding a new molecular building block to the synthetic molecules on the surface of the carrier bead and attaching labelling beads of the matching combination of types to the surface of the carrier bead at each step of combinatorial synthesis to form carrier beads with synthetic molecules 2, 3, 4, . . . having, respectively, 2, 3, 4, . . . molecular building blocks and with labelling beads from the cohorts, 2, 3, 4, . . . , respectively, and with the combination of labelling bead types matching the types of the molecular building blocks attached to the surface of the carrier bead at the corresponding steps of the synthesis; and
e) obtaining, as an end product, carrier beads compatible with an optical interrogation technique to detect all the types of labelling bead types attached to the surface of each carrier bead.

26. A method of decoding the composition of the synthetic molecules on carrier beads of claim 23, 24, or 25, the method comprising:
a) providing a population of carrier beads comprised of a plurality of groups, each group of the carrier beads in the population having a unique synthetic compound on the surface and unique fluorescent encoding provided by a unique set of types of labelling beads attached to the surface of the carrier beads;
b) optically interrogating each carrier bead of interest with an optical interrogation technique;
c) using the results of the optical interrogation to identify all individual types of the labelling beads on the surface of each carrier bead of interest;
d) matching the newly identified types of the labelling beads with the types of molecular building blocks to identify all individual molecular building blocks in the synthetic molecules on the carrier bead and the order in which these building blocks were added;
e) using the information on the type and order of the newly identified building blocks to obtain the structure of the synthetic molecules on the carrier bead.

27. A combinatorial library according to claims 20 and 24 comprising a plurality of carrier beads, wherein the synthetic molecule of the carrier beads is cleavable by light or chemically cleavable.

28. A combinatorial library according to claims 20 and 24 comprising a plurality of carrier beads wherein each carrier bead can be chemically or physically disintegrated, while the integrity of the labelling beads attached to the carrier beads is preserved.

29. A combinatorial library according to claims 20 and 24, wherein the synthetic molecules on the beads are macromolecules.

30. A method of screening a chemical library for molecules and macromolecules having certain desired properties, comprising:
(a) a population of carrier beads with a fluorescently encoded library of molecules or macromolecules of claims 20, 24, 27 and 28;
(b) assay testing the population of carrier beads for one or more testing outcomes;
(c) optically interrogating the population of carrier beads for which the desired testing outcomes were observed; and
(d) using the results of the optical interrogation to identify the molecular structures of the synthetic molecules on the carrier beads for which the desired testing outcome of the assay was observed.

31. A method according to claim 30, wherein the assay testing is performed in microwells, aqueous droplets, hydrogels, or living tissues.

32. A method according to claim 31, wherein the assay readout is biochemical, or lysate based, or cell based.

33. A method according to claim 32, wherein the assay information is obtained via sequencing or counting DNA or RNA molecules.
